(19) United States
(12) Patent Application Publication
(10) Pub. No.: US 2009/0143391 A1
(43) Pub. Date: Jun. 4, 2009

(54) Aryl and heteroaryl fused imidazo [1,5-a] pyrazines as inhibitors of phosphodiesterase 10

(76) Inventors: Norbert Hofgen, Ottendorf-Okrille (DE); Hans Stange, Riesa (DE); Barbara Langen, Radebeul (DE); Ute Egerland, Radebeul (DE); Rudolf Schindler, Dresden (DE)

Correspondence Address: FULBRIGHT & JAWORSKI, LLP, 666 FIFTH AVE, NEW YORK, NY, 10103-3198, US

(21) Appl. No.: 12/315,269

(22) Filed: Dec. 1, 2008

Related U.S. Application Data
(60) Provisional application No. 61/004,845, filed on Nov. 30, 2007.

(51) Int. Cl. A61K 31/4985; C07D 487/04; A61P 25/00; A61P 3/04

(52) U.S. Cl. 514/250; 544/346

(57) ABSTRACT

The invention relates to imidazo[1,5-a]pyrazine derivatives, to processes for preparing them, to pharmaceutical preparations which comprise these compounds and to the pharmaceutical use of these compounds, which are inhibitors of phosphodiesterase 10 (PDE10), as active compounds for treating central nervous system diseases of mammals, including humans.

### FIELD OF THE INVENTION

The invention relates to imidazo[1,5-a]pyrazine derivatives, to processes for preparing them, to pharmaceutical preparations which comprise these compounds and to the pharmaceutical use of these compounds, which are inhibitors of phosphodiesterase 10, as active compounds for treating diseases of mammals including a human which can be influenced by using the compounds according to the invention to inhibit phosphodiesterase 10 activity in the central nervous system. More particularly, the invention relates to the treatment of neurologic and psychiatric disorders, for example psychosis and disorders comprising cognitive deficits as symptoms.

### BACKGROUND

Psychotic disorders, especially schizophrenia, are severe mental disorders which extremely impair daily life. The symptoms of psychosis may be divided into two fractions. In the acute phase, it is predominated by hallucinations and delusions being called the positive symptoms. When the agitated phase abates the so called negative symptoms become obvious. They include cognitive deficits, social phobia, reduced vigilance, indifference and deficits in verbal learning and memory, verbal fluency and motor function.

Although several antipsychotics are available since, the present therapy of psychosis is not satisfactory. The classic antipsychotics, such as haloperidol, with a high affinity to dopamine D2 receptor show extreme side effects, such as extrapyramidal symptoms (=EPS) and do not improve the negative symptoms of schizophrenia so that they do not enable the patient to return to everyday life.

Clozapine which has emerged as a benchmark therapeutic ameliorating positive, negative and cognitive symptoms of schizophrenia and devoid of EPS shows agranulocytosis as a major, potential lethal side-effect (Capuano et al., Curr Med Chem 9: 521-548, 2002). Besides, there is still a high amount of therapy resistant cases (Lindenmayer et al., J Clin Psychiatry 63: 931-935, 2002).

In conclusion, there is still a need for developing new antipsychotics which ameliorate positive, negative and cognitive symptoms of psychosis and have a better side effect profile.

The exact pathomechanism of psychosis is not yet known. A dysfunction of several neurotransmitter systems has been shown. The two major neurotransmitter systems that are involved are the dopaminergic and the glutamatergic system:

Thus, acute psychotic symptoms may be stimulated by dopaminergic drugs (Capuano et al., Curr Med Chem 9: 521-548, 2002) and classical antipsychotics, like haloperidol, have a high affinity to the dopamine D2 receptor (Nyberg et al., Psychopharmacology 162: 37-41, 2002). Animal models based on a hyperactivity of the dopaminergic neurotransmitter system (amphetamine hyperactivity, apomorphine climbing) are used to mimic the positive symptoms of schizophrenia.

Additionally there is growing evidence that the glutamatergic neurotransmitter system plays an important role in the development of schizophrenia (Millan, Prog Neurobiol 70: 83-244, 2005). Thus, NMDA antagonists like phencyclidine and ketamine are able to stimulate schizophrenic symptoms in humans and rodents (Abi-Saab et al., Pharmacopsychiatry 31 Suppl 2: 104-109, 1998; Lahti et al., Neuropsychopharmacology 25: 455-467, 2001). Acute administration of phencyclidine and MK-801 induce hyperactivity, stereotypies and ataxia in rats mimicking psychotic symptoms. Moreover, in contrast to the dopaminergic models the animal models of psychosis based on NMDA antagonists do not only mimic the positive symptoms but also the negative and cognitive symptoms of psychosis (Abi-Saab et al., Pharmacopsychiatry 31 Suppl 2: 104-109, 1998; Jentsch and Roth, Neuropsychopharmacology 20: 201-225, 1999). Thus, NMDA antagonists, additionally induce cognitive deficits and social interaction deficits.

Eleven families of phosphodiesterases have been identified in mammals so far (Essayan, J Allergy Clin Immunol 108: 671-680, 2001). The role of PDEs in the cell signal cascade is to inactivate the cyclic nucleotides cAMP and/or cGMP (Soderling and Beavo, Proc Natl Acad USA 96(12):7071-7076, 2000). Since cAMP and cGMP are important second messenger in the signal cascade of G-protein-coupled receptors PDEs are involved in a broad range of physiological mechanisms playing a role in the homeostasis of the organism.

The PDE families differ in their substrate specificity for the cyclic nucleotides, their mechanism of regulation and their sensitivity to inhibitors. Moreover, they are differentially localized in the organism, among the cells of an organ and even within the cells. These differences lead to a differentiated involvement of the PDE families in the various physiological functions.

PDE10 (PDE10A) is primarily expressed in the brain and here in the nucleus accumbens and the caudate putamen. Areas with moderate expression are the thalamus, hippocampus, frontal cortex and olfactory tubercle (Menniti et al., William Harvey Research Conference, Porto, Dec. 6-8, 2001). All these brain areas are described to participate in the pathomechanism of schizophrenia (Lapiz et al., Neurosci Behav Physiol 33: 13-29, 2003) so that the location of the enzyme indicates a predominate role in the pathomechanism of psychosis.

In the striatum PDE10A is predominately found in the medium spiny neurons and they are primarily associated to the postsynaptic membranes of these neurons (Xie et al., Neuroscience 139: 597-607, 2006). By this location PDE10A may have an important influence on the signal cascade induced by dopaminergic and glutamatergic input on the medium spiny neurons two neurotransmitter systems playing a predominate role in the pathomechanism of psychosis.

Phosphodiesterase (PDE) 10A, in particular, hydrolyses both cAMP and cGMP having a higher affinity for cAMP (Kₘ=0.05 μM) than for cGMP (Kₘ=3 μM) (Soderling et al., Curr. Opin. Cell Biol 12: 174-179,1999).

Psychotic patients have been shown to have a dysfunction of cGMP and cAMP levels and its downstream substrates (Kaiya, Prostaglandins Leukot Essent Fatty Acids 46: 33-38,1992; Muly, Psychopharmacol Bull 36: 92-105, 2002; Garver et al., Life Sci 31: 1987-1992, 1982). Additionally, haloperidol treatment has been associated with increased cAMP and cGMP levels in rats and patients, respectively (Leveque et al., J Neurosci 20: 4011-4020, 2000; Gattaz et al., Biol Psychiatry 19: 1229-1235, 1984). As PDE10A hydrolyses both cAMP and cGMP (Kotera et al., Biochem Biophys Res Commun 261: 551-557, 1999), an inhibition of PDE10A would also induce an increase of cAMP and cGMP and thereby have a similar effect on cyclic nucleotide levels as haloperidol.

The antipsychotic potential of PDE10A inhibitors is further supported by studies of Kostowski et al. (Pharmacol Biochem Behav 5: 15-17, 1976) who showed that papaverine, a moderate selective PDE10A inhibitor, reduces apomorphine-induced stereotypies in rats, an animal model of psychosis, and increases haloperidol-induced catalepsy in rats while concurrently reducing dopamine concentration in rat brain, activities that are also seen with classical antipsychotics. This is further supported by a patent application establishing papaverine as a PDE10A inhibitor for the treatment of psychosis (US Patent Application Pub. No. 2003/0032579).

In addition to classical antipsychotics which mainly ameliorate the positive symptoms of psychosis, PDE10A also bears the potential to improve the negative and cognitive symptoms of psychosis.

Focusing on the dopaminergic input on the medium spiny neurons, PDE10A inhibitors by up-regulating cAMP and cGMP levels act as D1 agonists and D2 antagonists because the activation of Gs-protein coupled dopamine D1 receptor increases intracellular cAMP, whereas the activation of the Gi-protein coupled dopamine D2 receptor decreases intracellular cAMP levels through inhibition of adenylyl cyclase activity (Mutschler et al., Mutschler Arzneimittelwirkungen. 8^{th} ed. Stuttgart: Wissenschaftliche Verlagsgesellschaft mbH, 2001).

Elevated intracellular cAMP levels mediated by D1 receptor signalling seems to modulate a series of neuronal processes responsible for working memory in the prefrontal cortex (Sawaguchi, Parkinsonism Relat Disord 7: 9-19, 2000), and it is reported that D1 receptor activation may improve working memory deficits in schizophrenic patients (Castner et al., Science 287: 2020-2022, 2000). Thus, it seems likely that a further enhancement of this pathway might also improve the cognitive symptoms of schizophrenia.

Further indication of an effect of PDE10A inhibition on negative symptoms of psychosis was given by Rodefer et al. (Eur. J Neurosci 21: 1070-1076, 2005) who could show that papaverine reverses attentional set-shifting deficits induced by subchronic administration of phencyclidine, an NMDA antagonist, in rats. Attentional deficits including an impairment of shifting attention to novel stimuli belongs to the negative symptoms of schizophrenia. In the study the attentional deficits were induced by administering phencyclidine for 7 days followed by a washout period. The PDE10A inhibitor papaverine was able to reverse the enduring deficits induced by the subchronic treatment.

The synthesis of imidazo[1,5-a]pyrido[3,2-e]pyrazinones and some medical uses are well described in patents and the literature.

The documents EP 0 400 583 and U.S. Pat. No. 5,055,465 from Berlex Laboratories, Inc. report a group of imidazoquinoxalinones, their aza analogs and a process for their preparation. These compounds have been found to have inodilatory, vasodilatory and yenodilatory effects. The therapeutic activity is based on the inhibition of phosphodiesterase 3 (PDE3).

EP 0 736 532 reports pyrido[3,2-e]pyrazinones and a process for their preparation. These compounds are described to have anti-asthmatic and anti-allergic properties. Examples of this invention are inhibitors of PDE4 and PDE5.

WO 00/43392 reports the use of imidazo[I,5-a]pyrido[3,2-e]pyrazinones which are inhibitors of PDE3 and PDE5 for the therapy of erectile dysfunction, heart failure, pulmonic hypertonia and vascular diseases which are accompanied by insufficient blood supply.

Another group of pyrido[3,2-e]pyrazinones, reported in WO 01/68097 are inhibitors of PDE5 and can be used for the treatment of erectile dysfunction.

Further methods for the preparation of imidazo[I,5-a]pyrido[3,2-e]pyrazinones are described also by D. Norris et al. (Tetrahedron Letters 42 (2001 ), 4297-4299).

WO 92/22552 refers to imidazo[1,5-a]quinoxalines which are generally substituted at position 3 with a carboxylic acid group and derivatives thereof. These compounds are described to be useful as anxiolytic and sedativelhypnotic agents.

In contrast, only a limited number of imidazo[1,5-a]pyrido[3,2-e]pyrazines and their medical use are already published.

WO 99/45009 describes a group of imidazopyrazines of formula (I)

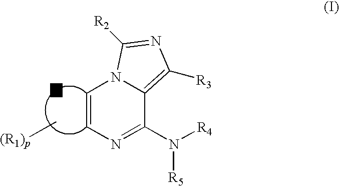

These compounds are described to be inhibitors of protein tyrosine kinases used in the treatment of protein tyrosine kinase-associated disorders such as immunologic disorders.

SAR data is reported in P. Chen et al., Bioorg. Med. Chem. Lett. 12 (2002), 1361-1364 and P. Chen et al., Bioorg. Med. Chem. Lett. 12 (2002), 3153-3156.

Imidazoquinoxalines with similar substituents are claimed in U.S. Pat. No. 6,235,740 B1. Again these compounds are described to be tyrosine kinase inhibitors that can be used for the treatment of e.g. immunologic disorders.

An other group of imidazoquinoxalines is claimed in U.S. Pat. No. 6,239,133 B1 were the amino substitution (U.S. Pat. No. 6,235,740 B1) is replaced by a number of substituents linked via oxygen, sulfur or a single bond. It is claimed that these compounds would also be useful for the treatment of immunologic diseases based on kinase inhibition.

### SUMMARY

The present invention provides compounds of formula (II):

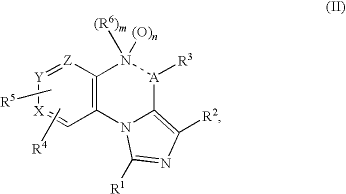

and pharmaceutical acceptable salts thereof, wherein variables R¹, R², R³, R⁴, R⁵, R⁶, X, Y, X, m, and n are as defined anywhere herein.

The present invention further provides compounds of formula (IIa),

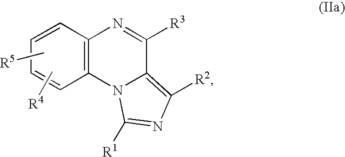

and pharmaceutical acceptable salts thereof, wherein variables R¹, R², R³, R⁴, and R⁵ are as defined anywhere herein.

The present invention further provides methods of preparing compounds of formula (II), wherein m and n are 0; the bond between A and N is a double bond; R³ is CN; and R¹, R², R⁴, and R⁵ are as defined anywhere herein.

The present invention provides methods of preparing compounds of formula (II),

wherein m and n are 0; the bond between A and N is a double bond;

R³ is selected from NHSO₂R⁸, N(SO₂R⁸)₂, N(R⁸)SO₂R⁸, NHSO₂R⁹, and N(R¹⁰)SO₂R⁹; and R¹, R², R⁴, R⁵, R⁸, and R⁹ are as defined anywhere herein.

The present invention further provides pharmaceutical compositions containing as an active agent one or more of the described compounds of the invention, or pharmaceutically acceptable salts thereof, optionally together with a pharmaceutically acceptable carrier.

The present invention further provides use of compounds of the invention, or pharmaceutically acceptable salts thereof, for the manufacture of a medicament for treating or preventing disorders associated with, accompanied by and/or caused by phosphodiesterase 10 hyperactivity and/or disorders.

The present invention further provides use of compounds of the invention, or pharmaceutically acceptable salts thereof, for the manufacture of a medicament for treating or preventing central nervous system disorders.

The present invention further provides use of compounds of the invention, or pharmaceutically acceptable salts thereof, for the manufacture of a medicament for improvement of learning and memory capacities in a mammal.

The present invention further provides use of compounds of the invention, or pharmaceutically acceptable salts thereof, for the manufacture of a medicament for treating or preventing obesity, type 2 diabetes, metabolic syndrome, or glucose intolerance.

The present invention further provides use of compounds of the invention, or pharmaceutically acceptable salts thereof, for the manufacture of a medicament for reducing body fat or body weight in a patient.

The present invention further provides pharmaceutical compositions or kits which contain at least one compound of the invention, or a pharmaceutically acceptable salt thereof, in combination with at least one further pharmaceutically active compound.

The present invention further provides compounds of the invention, or pharmaceutically acceptable salt thereof, for use in therapy.

The present invention further provides use of compounds of the invention, or pharmaceutically acceptable salt thereof, for the preparation of a medicament for use in therapy.

The details of one or more embodiments of the invention are set forth in the accompanying the description below. Other features, objects, and advantages of the invention will be apparent from the description and drawings, and from the claims.

### DETAILED DESCRIPTION

This invention relates to compounds of formula (II) and to pharmaceutically acceptable salts, solvates and derivatives such as prodrugs and metabolites thereof.

Compounds of formula (II)

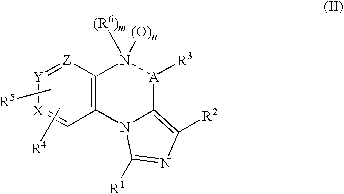

wherein the bond between A and N is a single bond or a double bond,
A is C when the bond is a double bond and CH when the bond is a single bond,
m is 0 or 1,
n is 0 or 1,
X, Y and Z are independently selected from C and N wherein not more than one of X, Y and Z can be N,
   wherein R¹ and R² are independently selected from
H, halo,
a cyclic radical,
C₁₋₈ alkyl, optionally mono- or polysubstituted with halo, OH, O—C₁₋₃ alkyl and/or a cyclic radical,
C₂₋₈ alkenyl, optionally mono- or polysubstituted with halo, OH, O—C₁₋₃ alkyl and/or a cyclic radical,
C₂₋₈ alkynyl, optionally mono- or polysubstituted with halo, OH, O—C₁₋₃-alkyl and/or a cyclic radical, and
a saturated, monounsaturated or polyunsaturated carbocyclic ring system with 3 to 8 ring atoms, e.g. phenyl, or a heterocyclic ring system with 5 to 15 ring atoms containing at least one heteroatom selected from N including N-oxide, O and S, each optionally mono- or polysubstituted with halo, amino, C₁₋₃ alkylamino, di-C₁₋₃ alkylamino, nitro, C₁₋₃ alkyl, O—C₁₋₃ alkyl, CF₃, COOH, CONH₂, CONHR⁷, CON(R⁷)₂ and/or a cyclic radical;
   wherein R⁷ is in each case independently C₁₋₆ alkyl, C₂₋₆ alkenyl, C₂₋₆ alkynyl, C₃₋₆ cycloalkyl, phenyl or a heterocyclic ring system with 5 to 6 ring atoms containing at least one heteroatom selected from N including N-oxide, O and S, each optionally mono- or polysubstituted with halo, OH, O—C₁₋₃ alkyl and/or a cyclic radical, or
   two R⁷ in group CON(R⁷)₂, together with the nitrogen atom to which they are attached, form a saturated or unsaturated five-, six- or seven-membered ring which can contain up to 3 heteroatoms selected from N,N-oxide, S and O, optionally mono- or polysubstituted with halo, C₁₋₃ alkyl, O—C₁₋₃ alkyl and/or aryl-C₁₋₅-alkyl wherein aryl is phenyl, optionally mono- or polysubstituted with halo, nitro, C₁₋₃ alkyl, and/or O—C₁₋₃ alkyl, and/or a cyclic radical;
   wherein R³ is selected from
H,
a cyclic radical,
N₃,
CN,
SOR⁸, SO₂R⁸,
NH(CO)OR⁸, N((CO)OR⁸)₂, NR⁸((CO)OR⁸),
NH—(C═O)—NH₂, NR⁸—(C═O)—NH₂,
NH—(C═O)—NHR⁸, NR⁸—(C═O)—NHR⁸,
NH—SO₂R⁸, N(SO₂R⁸)₂, and NR⁸(SO₂R⁸),
R⁹, NHSO₂R⁹, N(SO₂R⁹)₂, or N(R¹⁰)SO₂R⁹,
   wherein R⁸ is in each case independently,
a cyclic radical,
C₁₋₈ alkyl, C₃₋₈ cyclo(hetero)alkyl,
C₂₋₈ alkenyl, C₃₋₈ cyclo(hetero)alkenyl,
or C₂₋₈ alkynyl each optionally mono or polysubstituted with halo, OH and/or
O—C₁₋₃ alkyl, and/or a cyclic radical,
   wherein R⁹ is aryl, heteroaryl, aryl-C₁₋₅ alkyl, heteroaryl-C₁₋₅ alkyl, wherein aryl is phenyl or naphthyl, heteroaryl is an aromatic heterocyclic ring system of 5 to 15 ring atoms containing at least one atom selected from N including N-oxide, S, and O and wherein aryl and heteroaryl are optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical, and

R¹⁰ is C₁₋₅ alkyl, optionally mono or polysubstituted with halo, OH, O—C₁₋₃ alkyl and/or a cyclic radical,

wherein R⁴ and R⁵ in each case are independently selected from
H,
halo,
a cyclic radical,
R¹¹,
OH or OR¹¹,
NH(C═O)—C₁₋₃ alkyl, optionally mono- or polysubstituted with halo, OH, O—C₁₋₃
alkyl and/or a cyclic radical
NH₂, NHR¹¹, and NR¹¹R¹²,
   wherein R¹¹ and R¹² are independently selected from
   a cyclic radical,
   C₁₋₆ alkyl or C₃₋₆ cyclo(hetero)alkyl, optionally mono- or polysubstituted with halo, OH, O—C₁₋₃ alkyl and/or a cyclic radical,
   aryl-C₁₋₅-alkyl wherein aryl is phenyl, optionally mono- or polysubstituted with halo, amino, C₁₋₃ alkylamino, di-C₁₋₃ alkylamino, nitro, C₁₋₃ alkyl, OH, O—C₁₋₃ alkyl and/or a cyclic radical, or
   R¹¹ and R¹², together with the nitrogen atom to which they are attached, form a saturated or unsaturated five-, six- or seven-membered ring which can contain up to 3 heteroatoms selected from N,N-oxide, S and/or O, optionally mono- or polysubstituted with halo, amino, C₁₋₃ alkylamino, di-C₁₋₃ alkylamino, C₁₋₃ alkyl, O—C₁₋₃ alkyl and/or aryl-C₁₋₅-alkyl wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical, and
      wherein R⁶ is selected from H, C₁₋₅ alkyl, C₃₋₆ cycloalkyl, and (CO)—C₁₋₅ alkyl, optionally mono- or polysubstituted with halo, OH, O—C₁₋₃ alkyl and/or a cyclic radical,
      or pharmaceutically acceptable salts and derivatives thereof.

In some embodiments, compounds of the invention have formula (II)

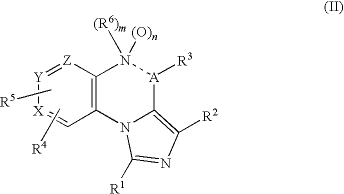

wherein the bond between A and N is a single bond or a double bond,
A is C when the bond is a double bond and CH when the bond is a single bond,
m is 0 or 1,
n is 0 or 1,
X, Y and Z are independently selected from C and N wherein not more than one of X, Y and Z can be N,
   wherein R¹ and R² are independently selected from
H, halo,
a cyclic radical,
C₁₋₈ alkyl, optionally mono- or polysubstituted with halo, OH, O—C₁₋₃ alkyl and/or a cyclic radical,
C₂₋₈ alkenyl, optionally mono- or polysubstituted with halo, OH, O—C₁₋₃ alkyl and/or a cyclic radical,
C₂₋₈ alkynyl, optionally mono- or polysubstituted with halo, OH, O—C₁₋₃-alkyl and/or a cyclic radical, and
a saturated, monounsaturated or polyunsaturated carbocyclic ring system with 3 to 8 ring atoms, e.g. phenyl, or a heterocyclic ring system with 5 to 15 ring atoms containing at least one heteroatom selected from N including N-oxide, O and S, each optionally mono- or polysubstituted with halo, amino, C₁₋₃ alkylamino, di-C₁₋₃ alkylamino, nitro, C₁₋₃ alkyl, O—C₁₋₃ alkyl, CF₃, COOH, CONH₂, CONHR⁷, CON(R⁷)₂ and/or a cyclic radical;
   wherein R⁷ is in each case independently C₁₋₆ alkyl, C₂₋₆ alkenyl, C₂₋₆ alkynyl, C₃₋₆ cycloalkyl, phenyl or a heterocyclic ring system with 5 to 6 ring atoms containing at least one heteroatom selected from N including N-oxide, O and S, each optionally mono- or polysubstituted with halo, OH, O—C₁₋₃ alkyl and/or a cyclic radical, or
   two R⁷ in group CON(R⁷)₂, together with the nitrogen atom to which they are attached, form a saturated or unsaturated five-, six- or seven-membered ring which can contain up to 3 heteroatoms selected from N,N-oxide, S and O, optionally mono- or polysubstituted with halo, C₁₋₃ alkyl, O—C₁₋₃ alkyl and/or aryl-C₁₋₅-alkyl wherein aryl is phenyl, optionally mono- or polysubstituted with halo, nitro, C₁₋₃ alkyl, and/or O—C₁₋₃ alkyl, and/or a cyclic radical;
R³ is selected from
H,
N₃,
CN,
SOR⁸, SO₂R⁸,
NH(CO)OR⁸, N((CO)OR⁸)₂, NR⁸((CO)OR⁸),
NH—(C═O)—NH₂, NR⁸—(C═O)—NH₂,
NH—(C═O)—NHR⁸, NR⁸—(C═O)—NHR⁸,
NH—SO₂R⁸, N(SO₂R⁸)₂, NR⁸(SO₂R⁸),
NHSO₂R⁹, N(SO₂R⁹)₂, and N(R¹⁰)SO₂R⁹.
   wherein R⁸ is in each case independently,
a cyclic radical,
C₁₋₈ alkyl, C₃₋₈ cyclo(hetero)alkyl,
C₂₋₈ alkenyl, C₃₋₈ cyclo(hetero)alkenyl,
or C₂₋₈ alkynyl each optionally mono or polysubstituted with halo, OH and/or
O—C₁₋₃ alkyl, and/or a cyclic radical,
   wherein R⁹ is aryl, heteroaryl, aryl-C₁₋₅ alkyl, heteroaryl-C₁₋₅ alkyl,
   wherein aryl is phenyl or naphthyl, heteroaryl is an aromatic heterocyclic ring system of 5 to 15 ring atoms containing at least one atom selected from N including N-oxide, S, and O and wherein aryl and heteroaryl are optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical, and

R¹⁰ is C₁₋₅ alkyl, optionally mono or polysubstituted with halo, OH, O—C₁₋₃ alkyl and/or a cyclic radical,

wherein R⁴ and R⁵ in each case are independently selected from
H,
halo,
a cyclic radical,
R¹¹,
OH or OR¹¹,
NH(C═O)—C₁₋₃ alkyl, optionally mono- or polysubstituted with halo, OH, O—C₁₋₃ alkyl and/or a cyclic radical
NH₂, NHR¹¹, and NR¹¹R¹²,
   wherein R¹¹ and R¹² are independently selected from
   a cyclic radical,
   C₁₋₆ alkyl or C₃₋₆ cyclo(hetero)alkyl, optionally mono- or polysubstituted with halo, OH, O—C₁₋₃ alkyl and/or a cyclic radical,
   aryl-C₁₋₅-alkyl wherein aryl is phenyl, optionally mono- or polysubstituted with halo, amino, C₁₋₃ alkylamino, di-C₁₋₃ alkylamino, nitro, C₁₋₃ alkyl, OH, O—C₁₋₃ alkyl and/or a cyclic radical, or
   R¹¹ and R¹², together with the nitrogen atom to which they are attached, form a saturated or unsaturated five-, six- or seven-membered ring which can contain up to 3 heteroatoms selected from N,N-oxide, S and/or O, optionally mono- or polysubstituted with halo, amino, C₁₋₃ alkylamino, di-C₁₋₃ alkylamino, C₁₋₃ alkyl, O—C₁₋₃ alkyl and/or aryl-C₁₋₅-alkyl wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical, and
      wherein R⁶ is selected from H, C₁₋₅ alkyl, C₃₋₆ cycloalkyl, and (CO)—C₁₋₅ alkyl, optionally mono- or polysubstituted with halo, OH, O—C₁₋₃ alkyl and/or a cyclic radical,
      or pharmaceutically acceptable salts and derivatives thereof.

In some embodiments, the bond between A and N is a double bond.

In some embodiments, m is 0 or n is 0 or m and n are both 0.

In some embodiments, X and Y are C and Z is N, X and Z are C and Y is N, or Y and Z are C and X is N. In other embodiments, X, Y and Z are carbon atoms.

In some embodiments, R¹ is selected from
H,
C₁₋₄ alkyl, particularly C₂₋₄ alkyl optionally mono- or polysubstituted with halo, OH, O—C₁₋₃ alkyl and/or a cyclic radical,
C₃—C₈ cycloalkyl, optionally mono- or polysubstituted with halo, OH, O—C₁₋₃ alkyl and/or a cyclic radical, and
phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical.

In further embodiments, R¹ is selected from C₂₋₄ alkyl, cyclohexyl or phenyl, optionally substituted.

In some embodiments, R² is H or C₁₋₄ alkyl, particularly methyl, optionally substituted.

In some embodiments, R² is hydrogen, CF₃, CHF₂, CH₂F, or a methyl-group.

In some embodiments, R² is other than H.

In some embodiments, neither of R¹ and R² is H.

In some embodiments, R³ is H, N₃, CN, SOR⁸, SO₂R⁸, NH—SO₂R⁸, N(SO₂R⁸)₂, NR⁸(SO₂R⁸), NHSO₂R⁹, N(SO₂R⁹)₂, or N(R¹⁰)SO₂R⁹.

In some embodiments, R³ is CN.

In some embodiments, R³ is NH—(C═O)OR⁸, particularly NH—(C═O)—OC₁₋₅ alkyl, optionally mono- or polysubstituted as indicated above.

In some embodiments, R³ is NH—SO₂R⁸, particularly NH—SO₂—C₁₋₅ alkyl, optionally mono-or polysubstituted as indicated above.

In some embodiments, R⁴ and/or R⁵ are selected from
H, halo, such as F, C₁₋₃ alkyl, O—C₁₋₃ alkyl, NH₂, NHC₁₋₃ alkyl wherein alkyl is optionally mono- or polysubstituted with halo, OH, O—C₁₋₃ alkyl, and/or a cyclic radical;
NH(C═O)—C₁₋₃ alkyl, optionally mono- or polysubstituted with halo, OH, O—C₁₋₃ alkyl, and/or a cyclic radical;
tetrahydropyrrolyl, pyrrolyl, pyrazolyl, imidazolyl, 1,2,3-triazolyl, 1,2,4-triazolyl, piperidinyl, morpholinyl, and piperazinyl, optionally mono- or polysubstituted with halo, OH, C₁₋₅ alkyl and/or O—C₁₋₃ alkyl, or aryl-C₁₋₅-alkyl wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl and/or a cyclic radical, for example

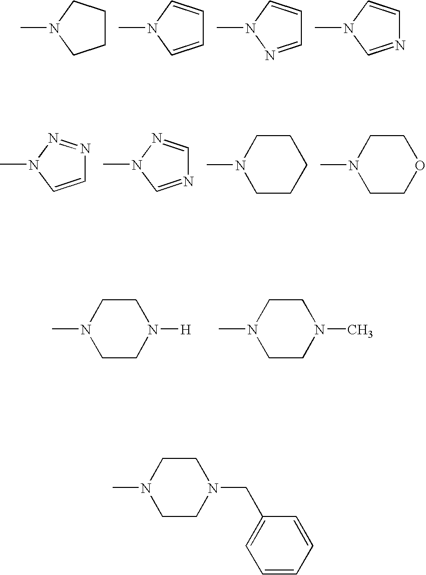

In some embodiments, R⁴ and R⁵ are selected from H, halo, C₁₋₃ alkyl, and O—C₁₋₃ alkyl, e.g. O-methyl, optionally substituted with a cyclic radical, e.g. a C₃₋₈ cycloalkyl. For example, R⁵ may be particularly H, F, Cl, OCH₃ or cyclopropylmethoxy (i.e. —O—CH₂-cyclopropyl).

In some embodiments, a substituent R⁴ or R⁵ different from H is located at positions 6, 7 and/or 8 of the ring system, i.e. bound to Z, Y and/or 8. In other embodiments, a substituent R⁴ or R⁵ different from H is bound to position 8 of the ring system, i.e. bound to X.

The present invention also includes compounds of formula (IIa)

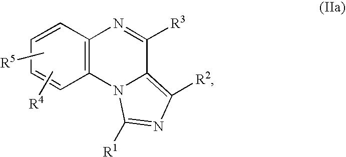

wherein
R¹ and R² are independently selected from
H, halo,
a cyclic radical,
C₁₋₈ alkyl optionally mono- or polysubstituted with halo, OH, O—C₁₋₃ alkyl, and/or a cyclic radical,
C₂₋₈ alkenyl optionally mono- or polysubstituted with halo, OH, O—C₁₋₃ alkyl, and/or a cyclic radical,
C₂₋₈ alkynyl optionally mono- or polysubstituted with halo, OH, O—C₁₋₃-alkyl and/or a cyclic radical,
a saturated, monounsaturated or polyunsaturated carbocyclic ring system with 3 to 8 atoms or a heterocyclic ring system with 5 to 15 ring atoms containing at least one heteroatom selected from N,N-oxide, O, and S, each optionally mono- or polysubstituted with halo, amino, C₁₋₃ alkylamino, di-C₁₋₃ alkylamino, nitro, C₁₋₃ alkyl, O—C₁₋₃ alkyl, CF₃, COOH, CONH₂, CONHR⁷, CON(R⁷)₂, or a cyclic radical;

R⁷ is C₁₋₆ alkyl, C₂₋₆ alkenyl, C₂₋₆ alkynyl, C₃₋₆ cycloalkyl, phenyl or a heterocyclic ring system with 5 to 6 ring atoms containing at least one heteroatom selected from N,N-oxide, O, and S, each optionally mono- or polysubstituted with halo, OH, O—C₁₋₃ alkyl, and/or a cyclic radical; or two R⁷ in group CON(R⁷)₂, together with the nitrogen atom to which they are attached, form a saturated or unsaturated five-, six- or seven-membered ring which contains up to 3 heteroatoms selected from N,N-oxide, S, and O, optionally mono- or polysubstituted with halo, C₁₋₃ alkyl, O—C₁₋₃ alkyl, and/or aryl-C₁₋₅-alkyl wherein aryl is phenyl, optionally mono- or polysubstituted with halo, nitro, C₁₋₃ alkyl, O—C₁₋₃ alkyl, and/or a cyclic radical;
R³ is selected from
H,
N₃,
CN,
SOR⁸, SO₂R⁸,
NH(CO)OR⁸, N((CO)OR⁸)₂, NR⁸((CO)OR⁸),
NH—(C═O)—NH₂, NR⁸—(C═O)—NH₂,
NH—(C═O)—NHR⁸, NR⁸—(C═O)—NHR⁸,
NH—SO₂R⁸, N(SO₂R⁸)₂, NR⁸(SO₂R⁸),
NHSO₂R⁹, N(SO₂R⁹)₂, and N(R¹⁰)SO₂R⁹;

R⁸ is a cyclic radical, C₁₋₈ alkyl, C₃₋₈ cyclo(hetero)alkyl, C₂₋₈ alkenyl, C₃₋₈ cyclo(hetero)alkenyl, or C₂₋₈ alkynyl, each optionally mono or polysubstituted with halo, OH, O—C₁₋₃ alkyl, and/or a cyclic radical;

R⁹ is aryl, heteroaryl, aryl-C₁₋₅ alkyl, or heteroaryl-C₁₋₅ alkyl, wherein aryl is phenyl or naphthyl, heteroaryl is an aromatic heterocyclic ring system of 5 to 15 ring atoms containing at least one atom selected from N, N-oxide, S, and O and wherein aryl and heteroaryl are optionally mono- or polysubstituted with halo, amino, C₁₋₃ alkylamino, di-C₁₋₃ alkylamino, nitro, C₁₋₃ alkyl, O—C₁₋₃ alkyl, and/or a cyclic radical;

R¹⁰ is C₁₋₅ alkyl optionally mono or polysubstituted with halo, OH, O—C₁₋₃ alkyl, and/or a cyclic radical;

R⁴ and R⁵ in each case are independently selected from
H,
halo,
a cyclic radical,
R¹¹,
OH or OR¹¹,
NH(C═O)—C₁₋₃ alkyl optionally mono- or polysubstituted with halo, OH, O—C₁₋₃ alkyl, and/or a cyclic radical,
NH₂, NHR¹¹, and NR¹¹R¹²; and

R¹¹ and R¹² are independently selected from
a cyclic radical,
C₁₋₆ alkyl or C₃₋₆ cyclo(hetero)alkyl, optionally mono- or polysubstituted with halo, OH, O—C₁₋₃ alkyl, and/or a cyclic radical,
aryl-C₁₋₅ alkyl wherein aryl is phenyl, optionally mono- or polysubstituted with halo, amino, C₁₋₃ alkylamino, di-C₁₋₃ alkylamino, nitro, C₁₋₃ alkyl, OH, O—C₁₋₃ alkyl, and/or a cyclic radical,
or R¹¹ and R¹², together with the nitrogen atom to which they are attached, form a saturated or unsaturated five-, six- or seven-membered ring which contains up to 3 heteroatoms selected from N,N-oxide, S, and O, optionally mono- or polysubstituted with halo, amino, C₁₋₃ alkylamino, di-C₁₋₃ alkylamino, C₁₋₃ alkyl, O—C₁₋₃ alkyl, and/or aryl-C₁₋₅ alkyl wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl, and/or a cyclic radical;
or a pharmaceutically acceptable salt thereof.

In some embodiments, R¹ is C₁₋₈ alkyl optionally substituted with halo, O—C₁₋₃ alkyl, and/or a cyclic radical.

In some embodiments, R¹ is C₁₋₈ alkyl. In some embodiments, R¹ is ethyl or propyl.

In some embodiments, R¹ is a saturated, monounsaturated or polyunsaturated carbocyclic ring system with 3 to 8 atoms, optionally mono- or polysubstituted with halo, C₁₋₃ alkyl, and/or O—C₁₋₃ alkyl. In some embodiments, R¹ is cyclohexyl.

In some embodiments, R¹ is a polyunsaturated carbocyclic ring system with 3 to 8 atoms optionally mono- or polysubstituted with halo, C₁₋₃ alkyl, and/or O—C₁₋₃ alkyl.

In some embodiments, R¹ is phenyl optionally mono- or polysubstituted with halo, C₁₋₃ alkyl, and/or O—C₁₋₃ alkyl.

In some embodiments, R¹ is phenyl mono- or polysubstituted with halo, C₁₋₃ alkyl, and/or O—C₁₋₃ alkyl.

In some embodiments, R¹ is phenyl mono- or polysubstituted with fluoro, chloro, and/or methyl.

In some embodiments, R¹ is phenyl mono-substituted with chloro.

In some embodiments, R¹ is 2-chlorophenyl.

In some embodiments, R² is H or C₁₋₈ alkyl.

In some embodiments, R² is C₁₋₈ alkyl

In some embodiments, R² is methyl.

In some embodiments, R³ is H, N₃, CN, SOR⁸, SO₂R⁸, NH—SO₂R⁸, N(SO₂R⁸)₂, NR⁸(SO₂R⁸), NHSO₂R⁹, N(SO₂R⁹)₂, or N(R¹⁰)SO₂R⁹.

In some embodiments, R³ is CN.

In some embodiments, R³ is SO₂R⁸, and said R⁸ is C₁₋₈ alkyl.

In some embodiments, R³ is SO₂R⁸, and said R⁸ is methyl, ethyl, or propyl.

In some embodiments, R³ is NH—SO₂R⁸, NR⁸(SO₂R⁸), NHSO₂R⁹, or N(R¹⁰)SO₂R⁹.

In some embodiments, R³ is NH—SO₂R⁸, and said R⁸ is C₁₋₈ alkyl.

In some embodiments, R³ is NH—SO₂R⁸, and said R⁸ is methyl.

In some embodiments, each of R⁴ and R⁵ is independently selected from H, halo, C₁₋₃ alkyl, a cyclic radical, and O—C₁₋₃ alkyl, wherein O—C₁₋₃ alkyl is optionally mono- or polysubstituted with halo and/or a cyclic radical.

In some embodiments, one of R⁴ and R⁵ is halo, and the other of R⁴ and R⁵ is H.

In some embodiments, one of R⁴ and R⁵ is fluoro or chloro, and the other of R⁴ and R⁵ is H.

In some embodiments, one of R⁴ and R⁵ is O—C₁₋₃ alkyl optionally mono- or polysubstituted with halo or a cyclic radical.

In some embodiments, one of R⁴ and R⁵ is O—C₁₋₃ alkyl, and the other of R⁴ and R⁵ is H.

In some embodiments, one of R⁴ and R⁵ is OCH₃, and the other of R⁴ and R⁵ is H.

In some embodiments, one of R⁴ and R⁵ is O—C₁₋₃ alkyl mono-substituted with a cyclic radical, and the other of R⁴ and R⁵ is H. For example, the cyclic radical is cyclopropyl. In some embodiments, the cyclic radical is quinolinyl.

In some embodiments, R⁴ and R⁵ is O—C₁₋₃ alkyl polysubstituted with halo, and the other of R⁴ and R⁵ is H.

In some embodiments, one of R⁴ and R⁵ is O—CH₂CF₃, and the other of R⁴ and R⁵ is H.

In some embodiments, one of R⁴ and R⁵ is a cyclic radical, and the other of R⁴ and R⁵ is H. For example, the cyclic radical is piperidinyl.

In some embodiments, the invention includes compounds of formula (IIb)

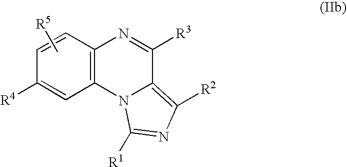

wherein R¹, R², R³, R⁴, and R⁵ are as defined anywhere herein.

In some embodiments, the invention includes compounds of formula (IIa)

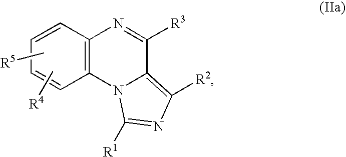

wherein
R¹ is C₁₋₈ alkyl, C₃₋₈ cycloalkyl, or phenyl mono-substituted with halo;
R² is C₁₋₈ alkyl;
R³ is CN or NH—SO₂R⁸, wherein R⁸ is C₁₋₈ alkyl; and
R⁴ and R⁵ in each case are independently selected from H, halo, C₃₋₆ cyclo(hetero)alkyl, or OR¹¹, wherein R¹¹ is C₁₋₆ alkyl optionally mono- or polysubstituted with halo and/or a cyclic radical;
or a pharmaceutically acceptable salt thereof.

Examples of specific compounds of the formula (II) are the following:
N-(1-Ethyl-3-methyl-imidazo(1,5-a)quinoxalin-4-yl)-methansulfonamide
N-(1-Ethyl-8-fluoro-3-methyl-imidazo(1,5-a)quinoxalin-4-yl)-methanesulfonamide
N-(8-Fluoro-3-methyl-1-propyl-imidazo(1,5-a)quinoxalin-4-yl)-methanesulfonamide
N-(1-(2-Chlorphenyl)-8-fluoro-3-methyl-imidazo(1,5-a)quinoxalin-4-yl)-methanesulfonamide
N-(1-Cyclohexyl-8-fluoro-3-methyl-imidazo(1,5-a)quinoxalin-4-yl)-methanesulfonamide
N-[1-Ethyl-3-methyl-8-(piperidin-1-yl)-imidazo(1,5-a)quinoxalin-4-yl]-methanesulfonamide
8-Fluoro-3-methyl-1-propyl-imidazo(1,5-a)quinoxaline-4-carbonitrile 1-Cyclohexyl-8-methoxy-3-methyl-imidazo[1,5-a]quinoxaline-4-carbonitrile;
N-(8-Methoxy-3-methyl-1-propyl-imidazo[1,5-a]quinoxalin-4-yl)-methanesulfonamide;
N-(1-Cyclohexyl-8-methoxy-3-methyl-imidazo[1,5-a]quinoxalin-4-yl)-methanesulfonamide;
N-(8-Cyclopropylmethoxy-3-methyl-1-propyl-imidazo[1,5-a]quinoxalin-4-yl)-methanesulfonamide;
N-(1-Cyclohexyl-8-cyclopropylmethoxy-3-methyl-imidazo[1,5-a]quinoxalin-4-yl)-methanesulfonamide;
N-[1-Cyclohexyl-3-methyl-8-(quinolin-2-ylmethoxy)-imidazo[1,5-a]quinoxalin-4-yl]-methanesulfonamide;
N-[1-(2-Chloro-phenyl)-7-methoxy-3-methyl-imidazo[1,5-a]quinoxalin-4-yl]-methanesulfonamide; and
N-(7-Methoxy-3-methyl-1-propyl-imidazo[1,5-a]quinoxalin-4-yl)-methanesulfonamide;
or pharmaceutically salts and derivatives thereof.

The present invention also provides a method of preparing a compound of formula (II), in which m and n are 0; the bond between A and N is a double bond; R³ is CN, and R¹, R², R⁴, and R⁵ are as defined anywhere herein; comprising reacting a compound of formula (IV)

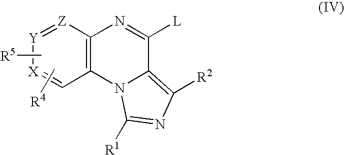

wherein L is Cl or Br; and R¹, R², R⁴, and R⁵ are as defined anywhere herein; with a cyanide salt. In some embodiments, the cyanide salt is KCN.

The present invention also provides a method of preparing a compound of formula (II), in which m and n are 0; the bond between A and N is a double bond; R³ is selected from NHSO₂R⁸, N(SO₂R⁸)₂, N(R⁸)SO₂R⁸, NHSO₂R⁹, and N(R¹⁰)SO₂R⁹; and
R⁸, R⁹ and R¹⁰ are as defined anywhere herein, comprising
(a) reacting a compound of formula (IV)

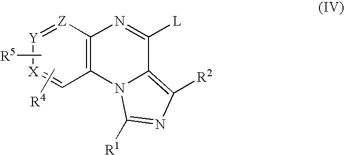

wherein L is Cl or Br; and R¹, R², R⁴, and R⁵ are as defined anywhere herein; with NH₃ or an alkyl amine to form a 4-amino derivative; and
(b) followed by reacting the 4-amino derivative with a sulfonic acid chloride or an anhydride to provide a final sulfonamide.

### Definitions

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “halo” refers to fluoro, chloro, bromo or iodo.

The terms “alkyl”, “alkenyl” and “alkynyl” refer to straight or branched hydrocarbon radicals with up to 8 carbon atoms preferably up to 6 carbon atoms and more preferably up to 5 carbon atoms such as methyl, ethyl, vinyl, ethynyl, propyl, isopropyl, allyl, propynyl, butyl, isobutyl, t-butyl, butenyl, butynyl etc. which may optionally be substituted as indicated above. “Alkyl” groups are saturated; an “alkenyl” group contains at least one double carbon-carbon bond; and an “alkynyl” group contains at least one triple carbon-carbon bond.

As used herein, “cyclic radical” refers to a saturated, unsaturated, or aromactic carbocycle or heterocycle, optionally mono- or polysubstituted with halo, amino, C₁₋₃ alkylamino, di-C₁₋₃ alkylamino, nitro, C₁₋₃ alkyl, OH, and/or O—C₁₋₃ alkyl. The cyclic radical can be a 3 to 24 membered mono- or polycyclic ring. In some embodiments, the cyclic radical is a 3-, 4-, 5-, 6-, or 7-membered ring. The cyclic radical can contain 3 to 20, or in some embodiments, 4 to 10 ring forming carbon atoms. The cyclic radical includes cyclo(hetero)alkyl, aryl and heteroaryl groups as defined below. “Cyclo(hetero)alkyl” refers to both cycloalkyl and cycloheteroalkyl groups. Cycloheteroalkyl and heteroaryl groups may, for example, contain 1 to 6, or in some embodiments, 1 to 3 ring forming heteroatoms, selected from O, N, S, and/or P. The cyclic radical can be bound via a carbon atom or optionally via a N, O, S, SO, or SO₂ group. An example of an aryl cyclic radical is phenyl. Examples of cycloalkyl cyclic radicals include cyclopropyl, cyclobutyl, cyclopentyl, cyclohexyl, and cycloheptyl. Examples of heteroaryl cyclic radicals include thienyl, furanyl, pyrroly, imidazolyl, triazolyl, oxazolyl, isoxazoly, pyrazolyl, thiazolyl, pyridinyl, pyrimidinyl, and the like. Examples of cycloheteroalkyl cyclic radicals include pyrrolidinyl, tetrahydrofuranyl, morpholino, thiomorpholino, piperazinyl, tetrahydrothienyl, 2,3-dihydrobenzofuryl, 1,3-benzodioxole, benzo-1,4-dioxane, piperidinyl, isoxazolidinyl, isothiazolidinyl, pyrazolidinyl, oxazolidinyl, thiazolidinyl, and imidazolidinyl. Examples of heteroaryl groups are provided below.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, and the like. In some embodiments, an aryl group has from 6 to about 20 carbon atoms.

As used herein, “arylalkyl” refers to an alkyl group substituted by an aryl group. Example arylalkyl groups include benzyl and phenylethyl.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of cyclopentane, cyclopentene, cyclohexane, and the like. A cycloalkyl group having one or more fused aromatic rings can be attached through either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like.

As used herein, a “heteroaryl” group refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, a “heteroarylalkyl” group refers to an alkyl group substituted by a heteroaryl group. An example of a heteroarylalkyl group is pyridylmethyl.

As used herein, “cycloheteroalkyl” refers to a non-aromatic heterocycle where one or more of the ring-forming atoms is a heteroatom such as an O, N, or S atom. Cycloheteroalkyl groups can include mono- or polycyclic (e.g., having 2, 3 or 4 fused rings) ring systems as well as spirocycles. Example cycloheteroalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of cycloheteroalkyl are moieties that have one or more aromatic rings fused (i.e., having a bond in common with) to the nonaromatic heterocyclic ring, for example phthalimidyl, naphthalimidyl, and benzo derivatives of heterocycles. A cycloheteroalkyl group having one or more fused aromatic rings can be attached though either the aromatic or non-aromatic portion. Also included in the definition of cycloheteroalkyl are moieties where one or more ring-forming atoms is substituted by 1 or 2 oxo or sulfido groups. In some embodiments, the cycloheteroalkyl group has from 1 to about 20 carbon atoms, and in further embodiments from about 3 to about 20 carbon atoms. In some embodiments, the cycloheteroalkyl group contains 3 to about 20, 3 to about 14, 3 to about 7, or 5 to 6 ring-forming atoms. In some embodiments, the cycloheteroalkyl group has 1 to about 4, 1 to about 3, or 1 to 2 heteroatoms. In some embodiments, the cycloheteroalkyl group contains 0 to 3 double bonds. In some embodiments, the cycloheteroalkyl group contains 0 to 2 triple bonds.

As used herein, the term “substituted” refers to the replacement of a hydrogen moiety with a non-hydrogen moiety in a molecule or group. A molecule or group may be monosubstituted. A molecule or group may be also polysubstituted with the same or different substituents. A substituent may be comprised of a single non-hydrogen moiety or of a combination of more than one non-hydrogen moieties, e.g. of halo and C₁₋₃ alkyl, thus being a C₁₋₃ halo alkyl substituent.

The term “reacting” is meant to refer to the bringing together of the indicated reagents in such a way as to allow their molecular interaction and chemical transformation according to the thermodynamics and kinetics of the chemical system. Reacting can be facilitated, particularly for solid reagents, by using an appropriate solvent or mixture of solvents in which at least one of the reagents is at least partially soluble. Reacting is typically carried out for a suitable time and under conditions suitable to bring about the desired chemical transformation.

The invention furthermore relates to the physiologically acceptable salts, solvates and derivatives of the compounds according to formula (II). Derivatives of the compounds according to formula (II) are, for example, amides, esters and ethers. Further, the term “derivative” also encompasses prodrugs and metabolites of compounds of formula (II).

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The physiologically acceptable salts may be obtained by neutralizing the bases with inorganic or organic acids or by neutralizing the acids with inorganic or organic bases. Examples of suitable inorganic acids are hydrochloric acid, sulphuric acid, phosphoric acid or hydrobromic acid, while examples of suitable organic acids are carboxylic acid, sulpho acid or sulphonic acid, such as acetic acid, tartaric acid, lactic acid, propionic acid, glycolic acid, malonic acid, maleic acid, fumaric acid, tannic acid, succinic acid, alginic acid, benzoic acid; 2-phenoxybenzoic acid, 2-acetoxybenzoic acid, cinnamic acid, mandelic acid, citric acid, maleic acid, salicylic acid, 3-aminosalicylic acid, ascorbic acid, embonic acid, nicotinic acid, isonicotinic acid, oxalic acid, gluconic acid, amino acids, methanesulphonic acid, ethanesulphonic acid, 2-hydroxyethanesulphonic acid, ethane-1,2-disulphonic acid, benzenesulphonic acid, 4-methylbenzenesulphonic acid or naphthalene-2-sulphonic acid. Examples of suitable inorganic bases are sodium hydroxide, potassium hydroxide and ammonia, while examples of suitable organic bases are amines, preferably, however, tertiary amines, such as trimethylamine, triethylamine, pyridine, N,N-dimethylaniline, quinoline, isoquinoline, α-picoline, β-picoline, γ-picoline, quinaldine and pyrimidine.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In addition, physiologically acceptable salts of the compounds according to formula (II) can be obtained by converting derivatives which possess tertiary amino groups into the corresponding quaternary ammonium salts in a manner known per se using quaternizing agents. Examples of suitable quaternizing agents are alkyl halides, such as methyl iodide, ethyl bromide and n-propyl chloride, and also arylalkyl halides, such as benzyl chloride or 2-phenylethyl bromide.

Furthermore, in the case of the compounds of the invention which contain an asymmetric carbon atom, the invention relates to the D form, the L form and D,L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, amide—imidic acid pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Compounds of the invention can also include all isotopes of atoms occurring in the intermediates or final compounds. Isotopes include those atoms having the same atomic number but different mass numbers. For example, isotopes of hydrogen include tritium and deuterium.

The term “compound” as used herein is meant to include all stereoisomers, geometric iosomers, tautomers, and isotopes of the structures depicted.

All compounds, and pharmaceuticaly acceptable salts thereof, are also meant to include solvated or hydrated forms.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof.

### Pharmaceutical Methods

The compounds according to the invention have been found to have pharmacologically important properties which can be used therapeutically. The compounds of the invention can be used alone, in combination with each other or in combination with other active compounds. The compounds according to the invention are inhibitors of phosphodiesterase 10. It is therefore a part of the subject-matter of this invention that the compounds of the invention and their salts and also pharmaceutical preparations which comprise these compounds or their salts, can be used for treating or preventing disorders associated with, accompanied by and/or covered by phosphodiesterase hyperactivity and/or disorders in which inhibiting phosphodiesterase 10 is of value.

Surprisingly, the compounds of the invention are potent inhibitors of the enzyme PDE10.

It is an embodiment of this invention, that compounds of the invention including their salts, solvates and prodrugs and also pharmaceutical compositions comprising an amount of a compound of the invention or one of its salts, solvates or prodrugs effective in inhibiting PDE10 can be used for the treatment of central nervous system disorders of mammals including a human.

More particularly, the invention relates to the treatment of neurological and psychiatric disorders including, but not limited to, (1) schizophrenia and other psychotic disorders; (2) mood [affective] disorders; (3) neurotic, stress-related and somatoform disorders including anxiety disorders; (4) eating disorders; sexual dysfunction comprising excessive sexual drive; (5) disorders of adult personality and behaviour; (6) disorders usually first diagnosed in infancy, childhood and adolescence; (7) mental retardation and (8) disorders of psychological development; (9) disorders comprising the symptom of cognitive deficiency in a mammal, including a human; (10) factitious disorders.

(1) Examples of schizophrenia and other psychotic disorders disorders that can be treated according to the present invention include, but are not limited to, continuous or episodic schizophrenia of different types (for instance paranoid, hebephrenic, catatonic, undifferentiated, residual, and schizophreniform disorders); schizotypal disorders (such as borderline, latent, prepsychotic, prodromal, pseudoneurotic pseudopsychopathic schizophrenia and schizotypal personality disorder); persistent delusional disorders; acute, transient and persistent psychotic disorders; induced delusional disorders; schizoaffective disorders of different type (for instance manic depressive or mixed type); puerperal psychosis and other and unspecified nonorganic psychosis.

(2) Examples of mood [affective] disorders that can be treated according to the present invention include, but are not limited to, manic episodes associated to bipolar disorder and single manic episodes, hypomania, mania with psychotic symptoms; bipolar affective disorders (including for instance bipolar affective disorders with current hypomanic and manic episodes with or without psychotic symptoms, bipolar I disorder or bipolar II disorder); depressive disorders, such as single episode or recurrent major depressive disorder of the mild moderate or severe type, depressive disorder with postpartum onset, depressive disorders with psychotic symptoms; persistent mood [affective] disorders, such as cyclothymia, dysthymia; premenstrual dysphoric disorder.

(3) Examples of disorders belonging to the neurotic, stress-related and somatoform disorders that can be treated according to the present invention include, but are not limited to, phobic anxiety disorders, for instance agoraphobia and social phobia primarily but not exclusively related to psychosis; other anxiety disorders such as panic disorders and general anxiety disorders; obsessive compulsive disorder; reaction to severe stress and adjustment disorders, such as post traumatic stress disorder; dissociative disorders and other neurotic disorders such as depersonalisation-derealisation syndrome.

(5) Examples of disorders of adult personality and behavior that can be treated according to the present invention include, but are not limited to, specific personality disorders of the paranoid, schizoid, schizotypal, antisocial, borderline, histrionic, narcissistic, avoidant, dissocial, emotionally unstable, anankastic, anxious and dependent type; mixed personality disorders; habit and impulse disorders (such as trichotillomania, pyromania, maladaptive aggression); disorders of sexual preference.

(6) Examples of disorders usually first diagnosed in infancy, childhood and adolescence that can be treated according to the present invention include, but are not limited to, hyperkinetic disorders, attentional deficit/hyperactivity disorder (AD/HD), conduct disorders; mixed disorders of conduct and emotional disorders; nonorganic enuresis, nonorganic encopresis; stereotyped movement disorder; and other specified behavioural emotional disorders, such as attention deficit disorder without hyperactivity, excessive masturbation nail-biting, nose-picking and thumb-sucking; disorders of psychological development particularly schizoid disorder of childhood and pervasive development disorders such as psychotic episodes associated to Asperger's syndrome.

Exemplary neurological disorders include neurodegenerative disorders including, without being limited to, Parkinson's disease, Huntington's disease, dementia (for example Alzheimer's disease, multi-infarct dementia, AIDS-related dementia, or fronto temperal dementia), neurodegeneration associated with cerebral trauma, neurodegeneration associated with stroke, neurodegeneration associated with cerebral infarct, hypoglycemia-induced neurodegeneration, neurodegeneration associated with epileptic seizure, neurodegeneration associated with neurotoxic poisoning or multi-system atrophy.

(8) Examples of disorders of psychological development include but are not limited to developmental disorders of speech and language, developmental disorders of scholastic skills, such as specific disorder of arithmetical skills, reading disorders and spelling disorders and other learning disorders. These disorders are predominantly diagnosed in infancy, childhood and adolescence.

(9) The phrase “cognitive deficiency” as used here in “disorder comprising as a symptom cognitive deficiency” refers to a subnormal functioning or a suboptimal functioning in one or more cognitive aspects such as memory, intellect, learning and logic ability, or attention in a particular individual comparative to other individuals within the same general age population.

Examples of disorders comprising as a symptom cognitive deficiency that can be treated according to the present invention include, but are not limited to, cognitive deficits primarily but not exclusively related to psychosis including schizophrenia, depression, age-associated memory impairment, autism, autistic spectrum disorders, fragile X syndrome, Parkinson's disease, Alzheimer's disease, multi infarct dementia, spinal cord injury, CNS hypoxia, Lewis body dementia, stroke, frontotemporal dementia, progressive supranuclear palsy Huntington's disease and in HIV disease, cerebral trauma, cardiovascular disease, drug abuse, diabetes associated cognitive impairment and mild cognitive disorder.

(11) Additionally, the invention relates to movement disorders with malfunction of basal ganglia. Examples of movement disorders with malfunction of basal ganglia that can be treated according to the present invention include, but are not limited to, different subtypes of dystonia, such as focal dystonias, multiple-focal or segmental dystonias, torsion dystonia, hemispheric, generalised and tardive dyskinesias (induced by psychopharmacological drugs), akathisias, dyskinesias such as Huntington's disease, Parkinson's disease, Lewis body disease, restless leg syndrome, PLMS.

(12) Furthermore the invention relates to the treatment of organic, including symptomatic mental disorders, especially to organic delusional (schizophrenia-like) disorders, presenil or senile psychosis associated to dementia, to psychosis in epilepsy and Parkinson's disease and other organic and symptomatic psychosis; delirium; infective psychosis; personality and behavioural disorders due to brain disease, damage and dysfunction.

(13) The invention relates to the treatment of mental and behavioural disorders due to psychoactive compounds, more particular to the treatment of psychotic disorders and residual and late-onset psychotic disorders induced by alcohol, opioids, cannabinoids, cocaine, hallucinogens, other stimulants, including caffeine, volatile solvents and other psychoactive compounds.

(14) The invention further relates to a general improvement of learning and memory capacities in a mammal, including a human.

Compounds currently used to treat schizophrenia have been associated with several undesirable side effects. These side effects include weight gain, hyperprolactinemia, elevated triglyceride levels, metabolic syndrome (markers: diabetes, hyperlipidemia, hypertension, and obesity), glucose abnormalities (such as hyperglycemia, elevated blood glucose and impaired glucose tolerance), and the exhibition of extrapyramidal symptoms. The weight gain observed with conventional atypical antipsychotics, such as risperidone and olanzapine, has been associated with an increased risk of cardiovascular disease and diabetes mellitus.

Compounds of the present invention are useful in treating schizophrenia to effect a clinically relevant improvement such as reduction of a PANSS total score in a patient, while maintaining body weight, maintaining or improving glucose levels and/or tolerance, maintaining and/or improving triglycerides levels and/or total cholesterol levels and/or maintaining an EPS profile similar to baseline measurements before administration.

The PDE10 inhibitors of the invention are further useful in the prevention and treatment of obesity, type 2 diabetes (non-insulin dependent diabetes), metabolic syndrome, glucose intolerance, and related health risks, symptoms or disorders. As such, the compounds can also be used to reduce body fat or body weight of an overweight or obese individual. In some embodiments, the PDE10 inhibitor is selective for PDE10, meaning that it is a better inhibitor of PDE10 than for any other PDE. In some embodiments, the selective PDE10 inhibitor can reduce PDE10 activity at least 10-fold or at least 100-fold compared to other PDE's.

As used herein, the terms “overweight” and “obese” are meant to refer to adult persons 18 years or older having a greater than ideal body weight (or body fat) measured by the body mass index (BMI). BMI is calculated by weight in kilograms divided by height in meters squared (kg/m²) or, alternatively, by weight in pounds, multiplied by 703, divided by height in inches squared (lbs×703/in²). Overweight individuals typically have a BMI of between 25 and 29, whereas obsess individuals typically have a BMI of 30 or more (see, e.g., National Heart, Lung, and Blood institute, Clinical Guidelines on the Identification, Evaluation, and Treatment of Overweight and Obesity in Adults, The Evidence Report, Washington, D.C.:U.S. Department of Health and Human Services, NIH publication no. 98-4083,1998). Other means for indicating excess body weight, excess body fat, and obesity include direct measure of body fat and/or waist-to-hip ratio measurements.

The term “metabolic syndrome” is used according to its usual meaning in the art. The American Heart Association characterizes metabolic syndrome as having at least 3 of the 5 below symptoms: 1) Elevated waist circumference (>102 cm (40 inches) in men; >88 cm (35 inches) in women), 2) Elevated triglycerides (>150 mg/dL (>1.7 mmol/L) or drug treatment for elevated triglycerides), 3) Reduced HDL-C (<40 mg/dL (1.03 mmol/L) in men <50 mg/dL (1.3 mmol/L) in women or drug treatment for reduced HDL-C, 4) Elevated blood pressure (>130/85 mmHg or drug treatment for hypertension), and 5) Elevated fasting glucose (>100 mg/dL or drug treatment for elevated glucose). See, Grundy, S. M. et al., Circulation, 2005, 112 (17, e285 (online at circ.ahajournals.org/cgi/reprint/112/17/e285)). Metabolic syndrome according to the World Health Organization (See, Alberti et al., Diabet. Med. 15, 539-553, 1998) includes individuals suffering from diabetes, glucose intolerance, low fasting glucose, or insulin resistance plus two or more of 1) High blood pressure (>160/90 mmHg), 2) Hyperlipdemia (triglycerides ≧150 mg/dL or HDL cholesterol <35 mg/dL in men and <39 mg/dL in women), 3) Central obesity (waist-to-hip ratio of >0.90 for men and >0.85 for women or BMI >30 kg/m²), and 4) Microalbuminuria (urinary albumin excretion rate ≧20 μg/min or an albumin-to-creatine ratio ≧20 μg/kg).

The present methods relating to reduction of body fat or body weight, as well as the treatment or prevention of obesity, type 2 diabetes (non-insulin dependent diabetes), metabolic syndrome, glucose intolerance, and related health risks, symptoms or disorders can be carried out by the administration of one or more compounds of the present invention. In some embodiments, one or more additional therapeutic agents can be administered such as anti-obesity agents. Example anti-obesity agents include apolipoprotein-B secretion/microsomal triglyceride transfer protein(apo-B/MTP) inhibitors, 11-beta-hydroxysteroid dehydrogenase-1 (11beta-HSD type 1) inhibitors, peptide YY3-36 or analogs thereof, MCR-4 agonists, cholecystokinin-A (CCK-A) agonists, monoamine reuptake inhibitors (such as sibutramine), cannabinoid receptor-I antagonists (such as rimona an, sympathomimetic agents, P3 adrenergic receptor agonists, 5 dopamine agonists; (such as bromocriptine), melanocyte-stimulating hormone receptor analogs, 5HT_{2C} agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), neuropeptide-Y receptor antagonists (e.g., NPY Y5 receptor antagonists, such as the compounds described in U.S. Pat. Nos. 6,566,367; 61/649,624; 61/638,942; 61/605,720; 61/495,569; 61/462,053; 61/388,077; 6,335,345; and 6,326,375; US Pat. Appl. Publ. Nos. 2002/0151456 and 20031036652; and PCT Publication Nos. WO 031010175, WO 03/082190 and receptor agonists or antagonists, orexin receptor antagonists, glucagon-like peptide-1 receptor agonists, ciliary neurotrophic factors, human agouti-related proteins (AGRP), ghrelin receptor antagonists, histamine 3 receptor antagonists or inverse agonists, neuromedin U receptor agonists and the like. Other anti-obesity agents are readily apparent to one of ordinary skill in the art.

Representative methods for using PDE10 inhibitors for the reduction of body fat or body weight, as well as the treatment or prevention of obesity, type 2 diabetes (non-insulin dependent diabetes), metabolic syndrome, glucose intolerance, and related health risks, symptoms are reported in WO 2005/120514.

The present invention also includes method of treating pain conditions and disorders. Examples of such pain conditions and disorders include, but are not limited to, inflammatory pain hyperalgesia, inflammatory hyperalgesia, migraine, cancer pain, osteoarthritis pain, post-surgical pain, non-inflammatory pain, neuropathic pain, sub-categories of neuropathic pain including peripheral neuropathic pain syndromes, chemotherapy-induced neuropathy, complex regional pain syndrome, HIV sensory neuropathy, neuropathy secondary to tumor infiltration, painful diabetic neuropathy, phantom limb pain, postherpetic neuralgia, postmastectomy pain, trigeminal neuralgia, central neuropathic pain syndromes, central poststroke pain, multiple sclerosis pain, Parkinson disease pain, and spinal cord injury pain.

In a further embodiment compounds of the present invention are administered in combination with one or more other agents effective for treating pain. Such agents include analgesics, non-steroidal anti-inflammatory drugs (NSAIDs), opiods and antidepressants. In various embodiments, one or more agents are selected from the group consisting of buprenorphine, naloxone, methadone, levomethadyl acetate, L-alpha acetylmethadol (LAAM), hydroxyzine, diphenoxylate, atropine, chlordiazepoxide, carbamazepine, mianserin, benzodiazepine, phenoziazine, disulfuram, acamprosate, topiramate, ondansetron, sertraline, bupropion, amantadine, amiloride, isradipine, tiagabine, baclofen, propranolol, tricyclic antidepressants, desipramine, carbamazepine, valproate, lamotrigine, doxepin, fluoxetine, imipramine, moclobemide, nortriptyline, paroxetine, sertraline, tryptophan, venlafaxine, trazodone, quetiapine, zolpidem, zopiclone, zaleplon, gabapentin, memantine, pregabalin, cannabinoids, tramadol, duloxetine, milnacipran, naltrexone, paracetamol, metoclopramide, loperamide, clonidine, lofexidine, and diazepam.

The present invention also includes methods of treating schizophrenia and other psychotic disorders, as described above, with a combination of compounds of the present invention with one or more antipsychotic agents. Examples of suitable antipsychotic agents for use in combination with the compounds of the present invention include, but are not limited to, the phenothiazine (chlorpromazine, mesoridazine, thioridazine, acetophenazine, fluphenazine, perphenazine and trifluoperazine), thioxanthine (chlorprothixene, thiothixene), heterocyclic dibenzazepine (clozapine, olanzepine and aripiprazole), butyrophenone (haloperidol), dipheyylbutylpiperidine (pimozide) and indolone (molindolone) classes of antipsychotic agents. Other antipsychotic agents with potential therapeutic value in combination with the compounds in the present invention include loxapine, sulpiride and risperidone.

The present invention further includes methods of treating depression or treatment-resistant depression with a combination of compounds of the present invention with one or more antidepressants. Examples of suitable anti-depressants for use in combination with the compounds of the present invention include, but are not limited to, norepinephrine reuptake inhibitors (tertiary and secondary amine tricyclics), selective serotonin reuptake inhibitors (SSRIs) (e.g., fluoxetine, fluvoxamine, paroxetine and sertraline), monoamine oxidase inhibitors (MAOIs) (isocarboxazid, phenelzine, tranylcypromine, selegiline), reversible inhibitors of monoamine oxidase (RIMAs) (moclobemide), serotonin and norepinephrine reuptake inhibitors (SNRIs) (venlafaxine), corticotropin releasing factor (CRF) receptor antagonists, alpha-adrenoreceptor antagonists, and atypical antidepressants (bupropion, lithium, nefazodone, trazodone and viloxazine).

### Compositions and Administration

An effective dose of the compounds according to the invention, or their salts, is used, in addition to physiologically acceptable carriers, diluents and/or adjuvants for producing a pharmaceutical composition. The dose of the active compounds can vary depending on the route of administration, the age and weight of the patient, the nature and severity of the diseases to be treated, and similar factors. The daily dose can be given as a single dose, which is to be administered once, or be subdivided into two or more daily doses, and is as a rule 0.001-2000 mg. Particular preference is given to administering daily doses of 0.1-500 mg, e.g. 0.1-100 mg.

Suitable administration forms are oral, parenteral, intravenous, transdermal, topical, inhalative, intranasal and sublingual preparations. Particular preference is given to using oral, parenteral, e.g. intravenous or intramuscular, intranasal preparations, e.g. dry powder or sublingual, of the compounds according to the invention. The customary galenic preparation forms, such as tablets, sugar-coated tablets, capsules, dispersible powders, granulates, aqueous solutions, alcohol-containing aqueous solutions, aqueous or oily suspensions, syrups, juices or drops, are used.

Solid medicinal forms can comprise inert components and carrier substances, such as calcium carbonate, calcium phosphate, sodium phosphate, lactose, starch, mannitol, alginates, gelatine, guar gum, magnesium stearate, aluminium stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavourings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators.

Examples of such additives are tartrate and citrate buffers, ethanol and sequestering agents (such as ethylenediaminetetraacetic acid and its non-toxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or water-miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Cellulose ethers which can dissolve or swell both in water or in organic solvents, such as hydroxypropylmethyl cellulose, methyl cellulose or ethyl cellulose, or soluble starches, can be used as film-forming agents.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allan-toin and novantisolic acid. Use of surfactants, emulsifiers or wetting agents, for example of Na lauryl sulphate, fatty alcohol ether sulphates, di-Na—N-lauryl-β-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

As indicated above, the compounds of the invention may be administered as a combination therapy with further active agents, e.g. therapeutically active compounds useful in the treatment of central nervous system disorders. These further compounds may be PDE10 inhibitors or compounds which have an activity which is not based on PDE10 inhibition such as dopamine D2 receptor modulating agents or NMDA modulating agents.

For a combination therapy, the active ingredients may be formulated as compositions containing several active ingredients in a single dose form and/or as kits containing individual active ingredients in separate dose forms. The active ingredients used in combination therapy may be co-administered or administered separately.

### Experiments

The synthesis of compounds of formula (II) can start from imidazo[1,5-a]pyrazinones of formula (III):

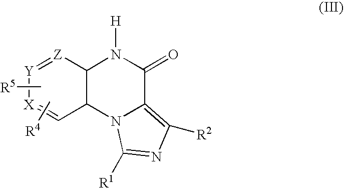

wherein R¹, R², R⁴, R⁵, X, Y, and Z are as described above.

The preparation of compounds of formula (III) is well described e.g. in J. Med. Chem. 1991,34,2671-2677.

According to standard procedures known from the literature and already used in WO 99/45009 compounds of formula (III) are halogenated by treatment with halogenating reagents like POCl₃, PCl₃, PCl₅ SOCl₂, POBr₃, PBr₃ or PBr₅, yielding e.g. 4-chloro or 4-bromo-imidazo[1,5-a]pyrazines of formula (IV).

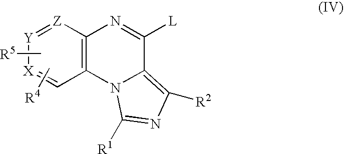

wherein L is Cl or Br and R¹, R², R⁴, R⁵, X, Y, and Z are as defined above.

### EXAMPLES

### Intermediate B1: 4-chloro-8-methoxy-3-methyl-1-propyl-imidazo[1,5-a]quinazoline

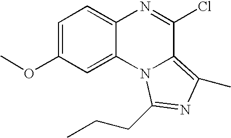

3.8 g of 8-methoxy-3-methyl-1-propyl-imidazo[1,5-a]quinazolin-4-one and 30 ml POCl₃ are mixed and heated up to reflux for 7 hours. After cooling to room temperature the reaction mixture is treated with 400 ml crushed ice/water and stirred for 1 hour. The product is extracted with 2×300 ml dichloromethane. The collected organic layer is washed with 300 ml water, 200 ml sodium carbonate solution (5%), 100 ml water, and dried with Na₂SO₄. The solvent is removed under reduced pressure.

Yield: 4.0 g

m.p.: 137-140° C.

### Intermediate B2: 4-Chloro-1-ethyl-3-methyl-8-piperidin-1 -yl-imidazo(1,5-a)quinoxaline

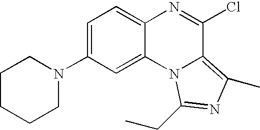

### 2-(2-Ethyl-4-methyl-imidazolyl)-4-piperidin-yl-nitrobenzene

5 g 2-(2-Ethyl-4-methyl-imidazolyl)-4-fluoro-nitrobenzene and 10 g piperidine were heated 30 minutes at 100° C. After cooling 150 ml ethylacetate were added. The solution was extracted three times with 50 ml water. The organic layer was evaporated to dryness. The residue is purified by chromatography (silica gel, dichloromethane/methanol=95/5).

Yield: 5.5 g

### 2-(2-Ethyl-4-methyl-imidazolyl)-4-piperidin-yl-aniline

5.0 g 2-(2-Ethyl-4-methyl-imidazolyl)-piperidin-yl-nitrobenzene were dissolved in 50 ml ethanol, and 0.5 g Pd/C 5% was added. The reaction was stirred 5 hours at 45° C. and 20 bar hydrogen. The catalyst was removed and the solution was evaporated to dryness.

Yield: 4.5 g

### 1-Ethyl-3-methyl-8-piperidin-yl-imidazo(1,5-a)quinoxalin-4-one

4.8 g 2-(2-Ethyl-4-methyl-imidazolyl)-4-piperidin-yl aniline and 16 g urea were heated 8 hours at 170° C. After cooling to 80° C. 80 ml water was added. After 1 hour stirring, the product is filtered off and dried at 60° C.

Yield: 4.2 g

m.p.: 313-317° C.

### 4-Chloro-1-ethyl-3-methyl-8-piperidin-1-yl-imidazo(1,5-a)quinoxaline

3.5 g 1-Ethyl-3-methyl-8-piperidin-1-yl-imidazo(1,5-a)quinoxalin-4-one were refluxed with 25 ml phosphoroxychloride for 8 hours. 25 ml toluene were added two times and destined to dryness. Then 100 ml ice water and 50 ml sodium carbonate solution (20%) were added. The mixture was extracted two times with 100 ml dichloromethane. The organic layer is evaporated to dryness, the residue was purified over silica gel, dichloromethane/methanol=95/5.

Yield: 0.99 g

m.p. 160-163° C.

Many other intermediates B of formula (IV) can be prepared according to this procedure. Some examples are the following:

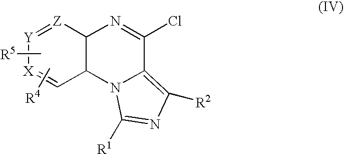

X, Y, Z=C

| Intermediate | R¹ | R² | R⁴ | R⁵ | m.p. [° C.] |
|---|---|---|---|---|---|
| B3 | —C₂H₅ | —CH₃ | 8-H | H | 125-128 |
| B4 | —C₃H₇ | —CH₃ | 8-H | H | 99-101 |
| B5 | —C₂H₅ | —CH₃ | 8-F | H | 157-160 |
| B6 | —C₃H₇ | —CH₃ | 8-F | H | 133-135 |
| B7 | -Cyclohexyl | —CH₃ | 8-F | H | 205-210 |
| B8 | —C₆H₄(2-Cl) | —CH₃ | 8-F | H | 189-190 |
| B9 | -Cyclohexyl | —CH₃ | 8-Cl | H | 244-248 |
| B10 | —C₃H₇ | —CH₃ | 8-Piperidin-1-yl | H | 105-108 |
| B11 | -Cyclohexyl | CH₃ | 8-OCH₃ | H | 212-215 |
| B12 | —C₃H₇ | CH₃ | 8-Cyclopropyl- | H | 103-105 |
| | | | methoxy | | |
| B13 | -Cyclohexyl | CH₃ | 8-Cyclopropyl- | H | 165-168 |
| | | | methoxy | | |
| B14 | —C₃H₇ | CH₃ | 8-(2,3,4-Trifluoro- | 6-(2,3,4- | 95-96 |
| | | | ethoxy) | Trifluoro- | |
| | | | | ethoxy) | |
| B15 | —C₃H₇ | CH₃ | 7-OCH₃ | H | 154-159 |
| B16 | —C₂H₅ | H | 8-Cl | H | 157-159 |

Compounds of formula (II) where m and n are 0, the bond between A and N is a double bond and R³ is —CN can be prepared by the treatment of an intermediate of formula (IV) with a cyanide salt, e.g. KCN.

### Example 1

### 8-Fluoro-3-methyl-1-propyl-imidazo(1,5-a)quinoxaline-4-carbonitrile

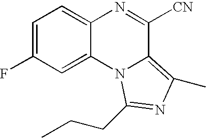

560 mg 4-Chloro-8-fluoro-3-methyl-1-propyl-imidazo(1,5-a)quinoxaline (2 mmol) were dissolved in 10 ml DMF and 600 mg potassium cyanide were added. The mixture was stirred 10 hours at 90 bis 100° C., cooled and 50 ml water and 50 ml toluene were added. The organic phase was washed two times with 25 ml water and distilled to dryness. The residue was chromatographed on silica gel with 50% ethylacetate, 45% dichloromethane, 5% methanol)

Yield: 0.31 g

m.p.: 187-188° C.

Compounds of formula (II) where m and n are 0, the bond between A and N is a double bond and R³ is NH—SO₂R⁶, N(SO₂R⁶)₂, N(R⁶)(SO₂R⁶), NHSO₂R⁷, N(SO₂R⁷)₂ and N(R⁸)SO₂R⁷ wherein R⁶, R⁷ and R⁸ are as defined above, can be prepared by treatment of an intermediate of formula (IV) with NH₃ or an alkyl amine, e.g. a C₁₋₅ alkyl amine to form the corresponding 4-amino derivatives according to the method from WO 99/45009. These 4-amino derivatives are treated with sulfonic acid chlorides or anhydrides forming the final sulfonamides.

### Example 2

### N-(1-Ethyl-3-methyl-imidazo(1,5-a)quinoxalin-4-yl)-methanesulfonamide

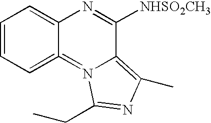

2.26 g 1-Ethyl-3-methyl-imidazo(1,5-a)quinoxalin-4-yl-amine (10 mmol) were stirred with 40 ml toluene. 2.18 g methanesulfonic acid anhydride (12.5 mmol) were added, and the mixture was heated 30 minutes under reflux. After cooling to 90° C. 3.0 g triethylamine were added. The reaction was stirred 15 minutes to 2 hours at 90-100° C., controlled with TLC. After cooling 25 ml water was added and 1 hour stirred at room temperature. The product was filtered off, washed two times with 20 ml water and 20 ml toluene, and dried.

Yield: 2.35 g

m.p. 195-199° C.

The dimethane sulfonated product can be converted into the Title Compound of Example 2 using MeOH/aqeous NaHCO₃ at 50-60° C.

Using the same procedure and reaction conditions like described above for Example 2 also following examples were synthesized.

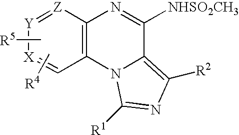

X, Y, Z=C

R⁵═H

| Example | R¹ | R² | R⁴ | m.p. [° C.] |
|---|---|---|---|---|
| 3 | Ethyl | Methyl | 8-F | 268-269 |
| 4 | Propyl | Methyl | 8-F | 277-280 |
| 5 | 2-Chlorophenyl | Methyl | 8-F | 249-253 |
| 6 | Cyclohexyl | Methyl | 8-F | 291-293 |
| 7 | Ethyl | Methyl | 8-Piperidin-1-yl | 223-227 |

Using the same procedure and reaction conditions as described above for Example 1, Example 8 was synthesized. Using the same procedure and reaction conditions as described above for Example 2, Examples 9-15 were synthesized.

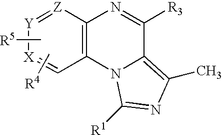

X, Y, Z=C

R⁵═H

| Example | R¹ | R³ | R⁴ | m.p. [° C.] |
|---|---|---|---|---|
| 8 | Cyclohexyl | CN | 8-OCH₃ | 244-248 |
| 9 | Propyl | NHSO₂CH₃ | 8-OCH₃ | 234-237 |
| 10 | Cyclohexyl | NHSO₂CH₃ | 8-OCH₃ | 287-288 |
| 11 | Propyl | NHSO₂CH₃ | 8-Cyclopropyl | 240-241 |
| | | | methoxy | |
| 12 | Cyclohexyl | NHSO₂CH₃ | 8-Cyclopropyl | 259-261 |
| | | | methoxy | |
| 13 | Cyclohexyl | NHSO₂CH₃ | 8-(Quinolin-2- | 255-258 |
| | | | ylmethoxy) | |
| 14 | 2-Chlorophenyl | NHSO₂CH₃ | 7-OCH₃ | 277-278 |
| 15 | Propyl | NHSO₂CH₃ | 7-OCH₃ | 222-226 |

Surprisingly, the compounds of formula (II) are potent inhibitors of the enzyme PDE10. A substance is considered to effectively inhibit PDE10 if it has an IC₅₀ of less than 10 μM, preferably less than 1 μM. IC₅₀ values for select compounds are provided in Table 1 below, where “+” indicates that the IC₅₀ value is less than or equal to 10 nM; “++” indicates that the IC₅₀ value is between 10-100 nM; and “+++” indicates that the IC₅₀ value is equal to or greater than 100 nM.

### Inhibition of PDE10

### Method A

Phosphodiesterase isoenzyme 10 (PDE10) activity was determined in preparations of rat, pig and guinea pig striatum respectively. Striatum from male Wistar rats (180-200 g), male hybrid pigs (150 kg) and male guinea pigs (CRL (HA), 500 g) respectively were collected and frozen at −70° C.

At the day of preparation 0.5 g striatum was homogenised in 10 ml 50 mM Tris/Mg-buffer at 4° C. and centrifuged for one hour at 100000 g. The supernatant is called the cytosolic fraction and was removed and stored on ice. The pellet was resuspended in the same buffer, but containing 1% Triton and incubated for 45 min at 4° C. Both fractions were independently applied onto a 5 ml Hi Trap™ QHP column at the Äkta-FPLC. After washing the columns the bound PDE protein was eluted with an increasing sodium chloride gradient (0 mM-500 mM sodium chloride) in 50 mM Tris/Mg-buffer at 4° C. for the cytosolic fraction and in the presence of 1% Triton for the membrane fraction. The eluted and collected fractions were tested with 100 nM [³H]-cAMP for PDE10-activity in the presence and without a specific PDE-Inhibitor at a concentration, were a 100% inhibition is expected. The fractions with PDE10-activity were pooled and frozen in aliquots until use at −20° C.

PDE10 activity was determined in a one step procedure in microtiter plates. The reaction mixture of 100 μl contained 50 mM Tris-HCl/5 mM MgCl₂ buffer (pH=7.4) (Sigma, Deisenhofen, Germany; Merck, Darmstadt, Germany) 0.1 μM [³H]-cAMP (Amersham, Buckinghamshire, UK) and the enzyme. Nonspecific activity was tested without the enzyme. The reaction was initiated by addition of the substrate solution and was carried out at 37 ° C. for 30 minutes. Enzymatic activity was stopped by addition of 25 μl YSi-SPA-beads (Amersham-Pharmacia). One hour later the mixture was measured in a liquid scintillation counter for microtiter plates (Microbeta Trilux). To pipette the incubation mixture a robot Biomek (Fa. Beckman) is used. The determined Km-values for the substrate cAMP is 78 nM for PDE10 from rat striatum, 88 nM for pig striatum and 66.7 nM for guinea pig striatum respectively. cGMP is the second substrate for PDE10. The Km values are 1800 nM, 2200 nM and 1700 nM for PDE10 from these species. For the test with cGMP 500 nM of this substrate was used. The optimal amount of enzyme in the assay has been determined and optimised for each enzyme preparation and substrate separately before using the enzyme in compound testing. For determination of IC₅₀ values the Hill-plot, 2-parameter-model, was used. Specific inhibitors of other PDE-subtypes do not inhibit the PDE10 preparation significantly. Papaverine was used as the most common PDE10 inhibitor and inhibits the PDE10 with IC₅₀ values of 142 nM, 110 nM and 77 nM for PDE10 from striatum of rat, pig and guinea pig respectively.

### Method B

Phosphodiesterase isoenzyme 10 (PDE10) activity was determined in preparations of human recombinant PDE10A and PDE10 from pig striatum respectively.

The DNA of PDE10A1 (AB 020593, 2340 bp) was synthesized and cloned into the vector pCR4.TOPO (Entelechon GmbH, Regensburg, Germany). The gene was than inserted into a baculovirus vector, ligated with the baculovirus DNA. The enzyme-protein was expressed in SF21-cells. The enzyme was isolated from these cells by harvesting the cells by a centrifugation at 200 g to collect the cells. The cells were resuspended in 50 mM Tris-HCl/5 mM MgCl₂ buffer (pH=7.4) and lysed by a sonication of the cells. The cytosolic PDE10A was obtained by a centrifugation at 48000 g for 1 h in the supernatant and stored at −70 ° C.

Striatum from male hybrid pigs (150 kg) were collected and frozen at −70° C. At the day of preparation 0.5 g striatum was homogenised in 10ml 50 mM Tris/Mg-buffer at 4° C. and centrifuged for one hour at 100000 g. The supernatant was removed and the pellet was resuspended in the same buffer, but containing 1% Triton and incubated for 45 min at 4° C. The membrane fraction was applied onto a 5 ml Hi Trap™ QHP column at the Äkta-FPLC. After washing the column the bound PDE protein was eluted with an increasing sodium chloride gradient (0 mM-500 mM sodium chloride) in 50 mM Tris/Mg-buffer at 4° C. in the presence of 1% Triton. The eluted and collected fractions were tested with 100 nM [3H]-cAMP for PDE10-activity in the presence and without a specific PDE-Inhibitor at a concentration, were a 100% inhibition is expected. The fractions with PDE10-activity were pooled and frozen in aliquots until use at −20° C.

PDE10 activity was determined in a one step procedure in microtiterplates. The reaction mixture of 100 μl contained 50 mM Tris-HCl/5 mM MgCl2 buffer (pH=7.4) (Sigma, Deisenhofen, Germany; Merck, Darmstadt, Germany) 0.1 μM [3H]-cAMP (Amersham, Buckinghamshire, UK) and the enzyme. Nonspecific activity was tested without the enzyme. The reaction was initiated by addition of the substrate solution and was carried out at 37° C. for 30 minutes. Enzymatic activity was stopped by addition of 25 μl YSi-SPA-beads (Amersham-Pharmacia). One hour later the mixture was measured in a liquid scintillation counter for microtiterplates (Microbeta Trilux). To pipette the incubation mixture a robot Biomek (Fa. Beckman) is used. The determined Km-values for the substrate cAMP is 88 nM for pig striatum and 130 nM for human recombinant PDE10A respectively. The optimal amount of enzyme in the assay has been determined and optimised for each enzyme preparation before using the enzyme in compound testing. For determination of IC50 values the Hill-plot, 2-parameter-model, was used. Specific inhibitors of other PDE-Subtypes do not inhibit the PDE10 preparation significantly. Papaverine was used as the most common PDE10 inhibitor and inhibits the PDE10 with IC50 values of 89 nM and 103 nM for PDE10 from human recombinant PDE10A and PDE10 from striatum of pig respectively.

The compounds according to this invention are potent inhibitors of the PDE10 with IC₅₀ values <1 μM.

The compounds of formula II show significant antipsychotic effects on the MK-801-induced hyperactivity and stereotyped sniffing, an animal model of psychosis.

### Test Procedure:

Female Wistar rats (Crl: (WI) BR, Charles River, Sulzfeld, Germany) weighing 150 to 180 g were used for the MK-801-induced psychosis. Animals were housed under standard conditions in groups of five on a 12 h light/dark cycle (light on at 0600 h) with ad libitum access to food (Pellets, ssniff M/R 15, Spezialdiät GmbH, Soest/Westfalen) and water.

MK-801 (dizocilpine, MW 337.37) was obtained by Tocris, distributed by Biotrend Chemikalien GmbH, Köln, Germany.

### Preparation of Compounds:

Compounds were freshly suspended in 0.5% hydroxyethylcellulose so that an administration volume of 0.5 ml/100 g was reached for each substance and dose. Hydroxyethylcellulose was solved in distilled water.

MK-801 was dissolved in saline so that an administration volume of 0.5 ml/100 g was reached. The suspensions and solutions were placed on a magnetic stirrer before and during dosing procedures.

The behaviour induced by the NMDA antagonist MK-801 is generally accepted as a rat model of psychosis. MK-801 induces stereotyped sniffing, hyperactivity and ataxia in rats after intraperitoneal administration.

Locomotor activity of the rats was recorded by the MotiTest Apparatus (TSE, Bad Homburg, Germany). The test area consisted of a squared arena (45×45 cm) with protective plexiglass walls (20 cm of height) where rats could freely move. Horizontal movements were recorded by 32 infrared photocells arranged along the bottom of each wall of the arena. The activity [sec] was measured by the computer program “ActiMot” (TSE, Bad Homburg, Germany).

Stereotyped sniffing was scored by the experimenter every five minutes for one hour (12 intervals) according to the method described by Andiné et al. (1999). The scores of the 12 intervals were summed up at the end of the recording time.

| Score | stereotyped sniffing |
|---|---|
| 0 | no stereotyped sniffing |
| 1 | discontinuous sniffing |
| | (free interval > 5 s) |
| 2 | continuous sniffing |

The day of experiment the female rats were placed in the laboratory and receive the test compound or vehicle at the appropriate time prior to test. MK-801 0.1 mg/kg was intraperitoneally administered 10 minutes prior to test.

At the beginning of the test the rats were placed in the centre of the squared arena of the MotiTest apparatus. Behaviour of the rats was recorded for one hour. After each run animals were removed and the boxes thoroughly cleaned and dried.

### Statistics:

Results were analysed by one way analysis of variance (ANOVA). Tukey test was used for individual comparison. P≦0.05 was regarded as significant.

Following p.o. or i.p. administration the compounds according to this invention demonstrate in vivo activity in this model at doses≦30 mg/kg.

TABLE 1
PDE 10 inhibition IC₅₀ values for select Examples

PDE10 inhibition
IC50 [nM]

| | | PDE10A |
|---|---|---|
| Example | PDE10 pig | human |
| B1 | | ++ |
| B2 | ++ | |
| B3 | ++ | |
| B4 | ++ | |
| B5 | ++ | |
| B6 | ++ | |
| B7 | ++ | |
| B8 | + | |
| B9 | +++ | |
| B10 | ++ | |
| B11 | | ++ |
| B12 | | ++ |
| B13 | | +++ |
| B14 | +++ | +++ |
| B15 | | ++ |
| B16 | | |
| 1 | ++ | |
| 2 | +++ | |
| 3 | +++ | |
| 4 | ++ | |
| 5 | ++ | |
| 6 | ++ | |
| 7 | ++ | |
| 8 | ++ | +++ |
| 9 | | ++ |
| 10 | | ++ |
| 11 | | + |
| 12 | + | ++ |
| 13 | + | + |
| 14 | ++ | ++ |
| 15 | ++ | ++ |

Various modifications of the invention, in addition to those described herein, will be apparent to those skilled in the art from the foregoing description. Such modifications are also intended to fall within the scope of the appended claims. Each reference, including all patents, patent applications, and journal literature, cited in the present application is incorporated herein by reference in its entirety.

1-81. (canceled)

82. A compound of formula (II) 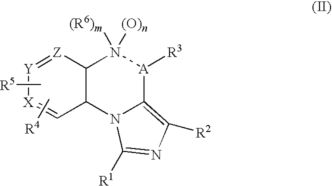
wherein
the bond between A and N is a single bond or a double bond;
A is C when the bond is a double bond and CH when the bond is a single bond;
m is 0 or 1;
n is 0 or 1;
X, Y and Z are independently selected from C and N wherein not more than one of X, Y and Z is N;
R¹ and R² are independently selected from
H, halo,
a cyclic radical,
C₁₋₈ alkyl optionally mono- or polysubstituted with halo, OH, O—C₁₋₃ alkyl, or a cyclic radical,
C₂₋₈ alkenyl optionally mono- or polysubstituted with halo, OH, O—C₁₋₃ alkyl, or a cyclic radical,
C₂₋₈ alkynyl optionally mono- or polysubstituted with halo, OH, O—C₁₋₃-alkyl, or a cyclic radical, a saturated, monounsaturated or polyunsaturated carbocyclic ring system with 3 to 8 ring atoms or a heterocyclic ring system with 5 to 15 ring atoms containing at least one heteroatom selected from N,N-oxide, O, and S, wherein each ring system is optionally mono- or polysubstituted with halo, amino, C₁₋₃ alkylamino, di-C₁₋₃ alkylamino, nitro, C₁₋₃ alkyl, O—C₁₋₃ alkyl, CF₃, COOH, CONH₂, CONHR⁷, CON(R⁷)₂, or a cyclic radical;
R⁷ is in each instance independently C₁₋₆ alkyl, C₂₋₆ alkenyl, C₂₋₆ alkynyl, C₃₋₆ cycloalkyl, phenyl or a heterocyclic ring system with 5 to 6 ring atoms containing at least one heteroatom selected from N,N-oxide, O, and S, each optionally mono- or polysubstituted with halo, OH, O—C₁₋₃ alkyl, or a cyclic radical,
or two R⁷ in group CON(R⁷)₂, together with the nitrogen atom to which they are attached, may form a saturated or unsaturated five-, six- or seven-membered ring which contains up to 3 heteroatoms selected from N,N-oxide, S, and O, optionally mono- or polysubstituted with halo, C₁₋₃ alkyl, O—C₁₋₃ alkyl, or aryl-C₁₋₅-alkyl wherein aryl is phenyl, optionally mono- or polysubstituted with halo, nitro, C₁₋₃ alkyl, O—C₁₋₃ alkyl, or a cyclic radical;
R³ is selected from
H,
N₃,
CN,
SOR⁸, SO₂R⁸,
NH(CO)OR⁸, N((CO)OR⁸)₂, NR⁸((CO)OR⁸),
NH—(C═O)—NH₂, NR⁸—(C═O)—NH₂,
NH—(C═O)—NHR⁸, NR⁸—(C═O)—NHR⁸,
NH—SO₂R⁸, N(SO₂R⁸)₂, NR⁸(SO₂R⁸),
NHSO₂R⁹, N(SO₂R⁹)₂, and N(R¹⁰)SO₂R⁹;
R⁸ is in each instance independently,
a cyclic radical,
C₁₋₈ alkyl, C₃₋₈ cyclo(hetero)alkyl,
C₂₋₈ alkenyl, C₃₋₈ cyclo(hetero)alkenyl,
or C₂₋₈ alkynyl, each optionally mono or polysubstituted with halo, OH, O—C₁₋₃ alkyl, or a cyclic radical;
R⁹ is aryl, heteroaryl, aryl-C₁₋₅ alkyl, or heteroaryl-C₁₋₅ alkyl,
wherein aryl is phenyl or naphthyl, heteroaryl is an aromatic heterocyclic ring system of 5 to 15 ring atoms containing at least one atom selected from N,N-oxide, S, and O and wherein aryl and heteroaryl are optionally mono- or polysubstituted with halo, amino, C₁₋₃ alkylamino, di-C₁₋₃ alkylamino, nitro, C₁₋₃ alkyl, O—C₁₋₃ alkyl, or a cyclic radical, and
R¹⁰ is C₁₋₅ alkyl optionally mono or polysubstituted with halo, OH, O—C₁₋₃ alkyl, or a cyclic radical,
R⁴ and R⁵ in each instance are independently selected from
H,
halo,
a cyclic radical,
R¹¹,
OH or OR¹¹,
NH(C═O)—C₁₋₃ alkyl optionally mono- or polysubstituted with halo, OH, O—C₁₋₃ alkyl, or a cyclic radical,
NH₂, NHR¹¹, and NR¹¹R¹²;
R¹¹ and R¹² are independently selected from
a cyclic radical,
C₁₋₆ alkyl or C₃₋₆ cyclo(hetero)alkyl, optionally mono- or polysubstituted with halo, OH, O—C₁₋₃ alkyl, or a cyclic radical,
aryl-C₁₋₅ alkyl wherein aryl is phenyl, optionally mono- or polysubstituted with halo, amino, C₁₋₃ alkylamino, di-C₁₋₃ alkylamino, nitro, C₁₋₃ alkyl, OH, O—C₁₋₃ alkyl, or a cyclic radical, or
or R¹¹ and R¹², together with the nitrogen atom to which R¹¹ and R¹² are attached, form a saturated or unsaturated five-, six- or seven-membered ring which contains up to 3 heteroatoms selected from N,N-oxide, S, and O, optionally mono- or polysubstituted with halo, amino, C₁₋₃ alkylamino, di-C₁₋₃ alkylamino, C₁₋₃ alkyl, O—C₁₋₃ alkyl, or aryl-C₁₋₅-alkyl wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl, or a cyclic radical; and
R⁶ is selected from
H,
C₁₋₅ alkyl, C₃₋₆ cycloalkyl, and (CO)—C₁₋₅ alkyl, optionally mono- or polysubstituted with halo, OH, O—C₁₋₃ alkyl, or a cyclic radical,
or a pharmaceutically acceptable salt thereof.

83. The compound of claim 82, or a pharmaceutically acceptable salt thereof, wherein the bond between A and N is a double bond.

84. The compound of claim 82, or a pharmaceutically acceptable salt thereof, wherein m and n are both 0.

85. The compound of claim 82, or a pharmaceutically acceptable salt thereof, wherein R¹ is C₂₋₄ alkyl, C₃—C₈ cycloalkyl, or phenyl, each optionally mono- or polysubstituted with halo or O—C₁₋₃ alkyl.

86. The compound of claim 82, or a pharmaceutically acceptable salt thereof, wherein R² is H, CF₃, CHF₂, CH₂F, or methyl.

87. The compound of claim 82, or a pharmaceutically acceptable salt thereof, wherein R³ is H, N₃, CN, SOR⁸, SO₂R⁸, NH—SO₂R⁸, N(SO₂R⁸)₂, NR⁸(SO₂R⁸), NHSO₂R⁹, N(SO₂R⁹)₂, or N(R¹⁰)SO₂R⁹.

88. The compound of claim 82, or a pharmaceutically acceptable salt thereof, wherein R³ is CN.

89. The compound of claim 82, or a pharmaceutically acceptable salt thereof, wherein R³ is —NH—(C═O)—OR⁸.

90. The compound of claim 82, or a pharmaceutically acceptable salt thereof, wherein R³ is —NH—SO₂R⁸.

91. The compound of claim 82, or a pharmaceutically acceptable salt thereof, wherein R⁴ and R⁵ are selected from H, halo, C₁₋₃ alkyl, and O—C₁₋₃ alkyl wherein alkyl is optionally mono- or polysubstituted with halo, OH, or O—C₁₋₃ alkyl.

92. A compound of formula (IIa) 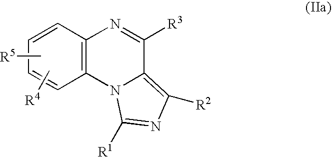
wherein
R¹ and R² are independently selected from
H, halo,
a cyclic radical,
C₁₋₈ alkyl optionally mono- or polysubstituted with halo, OH, O—C₁₋₃ alkyl, or a cyclic radical,
C₂₋₈ alkenyl optionally mono- or polysubstituted with halo, OH, O—C₁₋₃ alkyl, or a cyclic radical,
C₂₋₈ alkynyl optionally mono- or polysubstituted with halo, OH, O—C₁₋₃-alkyl or a cyclic radical,
a saturated, monounsaturated or polyunsaturated carbocyclic ring system with 3 to 8 atoms or a heterocyclic ring system with 5 to 15 ring atoms containing at least one heteroatom selected from N,N-oxide, O, and S, each optionally mono- or polysubstituted with halo, amino, C₁₋₃ alkylamino, di-C₁₋₃ alkylamino, nitro, C₁₋₃ alkyl, O—C₁₋₃ alkyl, CF₃, COOH, CONH₂, CONHR⁷, CON(R⁷)₂, or a cyclic radical;
R⁷ is C₁₋₆ alkyl, C₂₋₆ alkenyl, C₂₋₆ alkynyl, C₃₋₆ cycloalkyl, phenyl or a heterocyclic ring system with 5 to 6 ring atoms containing at least one heteroatom selected from N,N-oxide, O, and S, each optionally mono- or polysubstituted with halo, OH, O—C₁₋₃ alkyl, or a cyclic radical;
or two R⁷ in group CON(R⁷)₂, together with the nitrogen atom to which they are attached, form a saturated or unsaturated five-, six- or seven-membered ring which contains up to 3 heteroatoms selected from N,N-oxide, S, and O, optionally mono- or polysubstituted with halo, C₁₋₃ alkyl, O—C₁₋₃ alkyl, or aryl-C₁₋₅-alkyl wherein aryl is phenyl, optionally mono- or polysubstituted with halo, nitro, C₁₋₃ alkyl, O—C₁₋₃ alkyl, or a cyclic radical;
R³is selected from
H,
N₃,
CN,
SOR⁸, SO₂R⁸,
NH(CO)OR⁸, N((CO)OR⁸)₂, NR⁸((CO)OR⁸),
NH—(C═O)—NH₂, NR⁸—(C═O)—NH₂,
NH—(C═O)—NHR⁸, NR⁸—(C═O)—NHR⁸,
NH—SO₂R⁸, N(SO₂R⁸)₂, NR⁸(SO₂R⁸),
NHSO₂R⁹, N(SO₂R⁹)₂, and N(R¹⁰)SO₂R⁹;
R⁸ is a cyclic radical, C₁₋₈ alkyl, C₃₋₈ cyclo(hetero)alkyl, C₂₋₈ alkenyl, C₃₋₈ cyclo(hetero)alkenyl, or C₂₋₈ alkynyl, each optionally mono or polysubstituted with halo, OH, O—C₁₋₃ alkyl, or a cyclic radical;
R⁹ is aryl, heteroaryl, aryl-C₁₋₅ alkyl, or heteroaryl-C₁₋₅ alkyl,
wherein aryl is phenyl or naphthyl, heteroaryl is an aromatic heterocyclic ring system of 5 to 15 ring atoms containing at least one atom selected from N,N-oxide, S, and O and wherein aryl and heteroaryl are optionally mono- or polysubstituted with halo, amino, C₁₋₃ alkylamino, di-C₁₋₃ alkylamino, nitro, C₁₋₃ alkyl, O—C₁₋₃ alkyl, or a cyclic radical;
R¹⁰ is C₁₋₅ alkyl optionally mono or polysubstituted with halo, OH, O—C₁₋₃ alkyl, or a cyclic radical;
R⁴and R⁵ in each instance are independently selected from
H,
halo,
a cyclic radical,
R¹¹,
OH or OR¹¹,
NH(C═O)—C₁₋₃ alkyl optionally mono- or polysubstituted with halo, OH, O—C₁₋₃ alkyl, or a cyclic radical,
NH₂, NHR¹¹, and NR¹¹R¹²; and
R¹¹ and R¹² are independently selected from
a cyclic radical,
C₁₋₆ alkyl or C₃₋₆ cyclo(hetero)alkyl, optionally mono- or polysubstituted with halo, OH, O—C₁₋₃ alkyl, or a cyclic radical,
aryl-C₁₋₅ alkyl wherein aryl is phenyl, optionally mono- or polysubstituted with halo, amino, C₁₋₃ alkylamino, di-C₁₋₃ alkylamino, nitro, C₁₋₃ alkyl, OH, O—C₁₋₃ alkyl, or a cyclic radical,
or R¹¹ and R¹², together with the nitrogen atom to which R¹¹ and R¹² are attached, form a saturated or unsaturated five-, six- or seven-membered ring which contains up to 3 heteroatoms selected from N,N-oxide, S, and O, optionally mono- or polysubstituted with halo, amino, C₁₋₃ alkylamino, di-C₁₋₃ alkylamino, C₁₋₃ alkyl, O—C₃ alkyl, or aryl-C₁₋₅ alkyl wherein aryl is phenyl, optionally mono- or polysubstituted with halo, amino, C₁₋₃ alkylamino, di-C₁₋₃ alkylamino, nitro, C₁₋₃ alkyl, O—C₁₋₃ alkyl, or a cyclic radical;
or a pharmaceutically acceptable salt thereof.

93. The compound of claim 92, or a pharmaceutically acceptable salt thereof, wherein R¹ is C₁₋₈ alkyl optionally substituted with halo, O—C₁₋₃ alkyl, or a cyclic radical.

94. The compound of claim 92, or a pharmaceutically acceptable salt thereof, wherein R¹ is C₁₋₈ alkyl.

95. The compound of claim 94, or a pharmaceutically acceptable salt thereof, wherein R¹ is ethyl or propyl.

96. The compound of claim 92, or a pharmaceutically acceptable salt thereof, wherein R¹ is a saturated, monounsaturated or polyunsaturated carbocyclic ring system with 3 to 8 atoms, optionally mono- or polysubstituted with halo, C₁₋₃ alkyl, or O—C₁₋₃ alkyl.

97. The compound of claim 96, or a pharmaceutically acceptable salt thereof, wherein R¹ is cyclohexyl.

98. The compound of claim 96, or a pharmaceutically acceptable salt thereof, wherein R¹ is a polyunsaturated carbocyclic ring system with 3 to 8 atoms optionally mono- or polysubstituted with halo, C₁₋₃ alkyl, or O—C₁₋₃ alkyl.

99. The compound of claim 98, or a pharmaceutically acceptable salt thereof, wherein R¹ is phenyl optionally mono- or polysubstituted with halo, C₁₋₃ alkyl, or O—C₁₋₃ alkyl.

100. The compound of claim 98, or a pharmaceutically acceptable salt thereof, wherein R¹ is phenyl mono- or polysubstituted with halo, C₁₋₃ alkyl, or O—C₁₋₃ alkyl.

101. The compound of claim 100, or a pharmaceutically acceptable salt thereof, wherein R¹ is phenyl mono- or polysubstituted with fluoro, chloro, or methyl.

102. The compound of claim 101, or a pharmaceutically acceptable salt thereof, wherein
R¹ is phenyl mono-substituted with chloro.

103. The compound of claim 102, or a pharmaceutically acceptable salt thereof, wherein R¹ is 2-chlorophenyl.

104. The compound of claim 92, or a pharmaceutically acceptable salt thereof, wherein R² is H or C₁₋₈ alkyl.

105. The compound of claim 92, or a pharmaceutically acceptable salt thereof, wherein R² is C₁₋₈ alkyl

106. The compound of claim 105, or a pharmaceutically acceptable salt thereof, wherein R² is methyl.

107. The compound of claim 92, or a pharmaceutically acceptable salt thereof, wherein R³ is H, N₃, CN, SOR⁸, SO₂R⁸, NH—SO₂R⁸, N(SO₂R⁸)₂, NR⁸(SO₂R⁸), NHSO₂R⁹, N(SO₂R⁹)₂, or N(R¹⁰)SO₂R⁹.

108. The compound of claim 92, or a pharmaceutically acceptable salt thereof, wherein R³ is CN.

109. The compound of any one of claim 92, wherein R³ is SO₂R⁸.

110. The compound of claim 109, or a pharmaceutically acceptable salt thereof, wherein R⁸ is C₁₋₈ alkyl.

111. The compound of claim 109, or a pharmaceutically acceptable salt thereof, wherein R⁸ is methyl, ethyl, or propyl.

112. The compound of claim 92, or a pharmaceutically acceptable salt thereof, wherein R³ is NH—SO₂R⁸, NR⁸(SO₂R⁸), NHSO₂R⁹, or N(R¹⁰)SO₂R⁹.

113. The compound of claim 92, or a pharmaceutically acceptable salt thereof, wherein R³ is NH—SO₂R⁸.

114. The compound of claim 113, or a pharmaceutically acceptable salt thereof, wherein R⁸ is C₁₋₈ alkyl.

115. The compound of claim 114, or a pharmaceutically acceptable salt thereof, wherein R⁸ is methyl.

116. The compound of claim 92, or a pharmaceutically acceptable salt thereof, wherein each of R⁴ and R⁵ is independently selected from H, halo, C₁₋₃ alkyl, a cyclic radical, and O—C₁₋₃ alkyl, wherein O—C₁₋₃ alkyl is optionally mono- or polysubstituted with halo or a cyclic radical.

117. The compound of claim 92, or a pharmaceutically acceptable salt thereof, wherein one of R⁴ and R⁵ is halo, and the other of R⁴ and R⁵ is H.

118. The compound of claim 117, or a pharmaceutically acceptable salt thereof, wherein one of R⁴ and R⁵ is fluoro or chloro, and the other of R⁴ and R⁵ is H.

119. The compound of claim 32, or a pharmaceutically acceptable salt thereof, wherein one of R⁴ and R⁵ is O—C₁₋₃ alkyl optionally mono- or polysubstituted with halo or a cyclic radical.

120. The compound of claim 119, or a pharmaceutically acceptable salt thereof, wherein one of R⁴ and R⁵ is O—C₁₋₃ alkyl, and the other of R⁴ and R⁵ is H.

121. The compound of claim 120, or a pharmaceutically acceptable salt thereof, wherein one of R⁴ and R⁵ is OCH₃, and the other of R⁴ and R⁵ is H.

122. The compound of claim 119, or a pharmaceutically acceptable salt thereof, wherein one of R⁴ and R⁵ is O—C₁₋₃ alkyl mono-substituted with a cyclic radical, and the other of R⁴ and R⁵ is H.

123. The compound of claim 122, or a pharmaceutically acceptable salt thereof, wherein the cyclic radical is cyclopropyl.

124. The compound of claim 122, or a pharmaceutically acceptable salt thereof, wherein the cyclic radical is quinolinyl.

125. The compound of claim 119, or a pharmaceutically acceptable salt thereof, wherein one of R⁴ and R⁵ is O—C₁₋₃ alkyl polysubstituted with halo, and the other of R⁴ and R⁵ is H.

126. The compound of claim 125, or a pharmaceutically acceptable salt thereof, wherein one of R⁴ and R⁵ is O—CH₂CF₃, and the other of R⁴ and R⁵ is H.

127. The compound of claim 116, or a pharmaceutically acceptable salt thereof, wherein one of R⁴ and R⁵ is a cyclic radical, and the other of R⁴ and R⁵ is H.

128. The compound of claim 127, or a pharmaceutically acceptable salt thereof, wherein the cyclic radical is piperidinyl.

129. The compound of claim 92, or a pharmaceutically acceptable salt thereof, having formula (IIb) 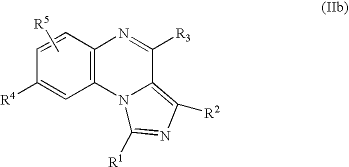
wherein R¹, R², R³, R⁴ and R⁵ are as defined in claim 82.

130. The compound of claim 82, or a pharmaceutically acceptable salt thereof, wherein the compound has formula (IIa) 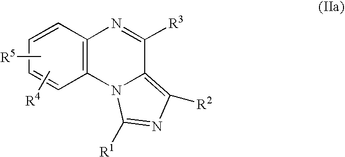
wherein
R¹ is C₁₋₈ alkyl, C₃₋₈ cycloalkyl, or phenyl mono-substituted with halo;
R² is C₁₋₈ alkyl;
R³ is CN or NH—SO₂R⁸, wherein R⁸ is C₁₋₈ alkyl; and
R⁴ and R⁵ in each instance are independently selected from H, halo, C₃₋₆ cyclo(hetero)alkyl, or OR¹¹, wherein R¹¹ is C₁₋₆ alkyl optionally mono- or polysubstituted with halo or a cyclic radical;
or a pharmaceutically acceptable salt thereof.

131. The compound of claim 82 selected from the group consisting of
N-(1-Ethyl-3-methyl-imidazo(1,5-a)quinoxalin-4-yl)-methanesulfonamide;
N-(1-Ethyl-8-fluoro-3-methyl-imidazo(1,5-a)quinoxalin-4-yl)-methanesulfonamide;
N-(8-Fluoro-3-methyl-1-propyl-imidazo(1,5-a)quinoxalin-4-yl)-methanesulfonamide;
N-(1-(2-Chlorphenyl)-8-fluoro-3-methyl-imidazo(1,5-a)quinoxalin-4-yl)-methanesulfonamide;
N-(1-Cyclohexyl-8-fluoro-3-methyl-imidazo(1,5-a)quinoxalin-4-yl)-methanesulfonamide;
N-[1-Ethyl-3-methyl-8-(piperidin-1-yl)-imidazo(1,5-a)quinoxalin-4-yl]-methansulfonamide;
8-Fluoro-3-methyl-1-propyl-imidazo(1,5-a)quinoxaline-4-carbonitrile;
1-Cyclohexyl-8-methoxy-3-methyl-imidazo[1,5-a]quinoxaline-4-carbonitrile;
N-(8-Methoxy-3-methyl-1-propyl-imidazo[1,5-a]quinoxalin-4-yl)-methanesulfonamide;
N-(1-Cyclohexyl-8-methoxy-3-methyl-imidazo[1,5-a]quinoxalin-4-yl)-methanesulfonamide;
N-(8-Cyclopropylmethoxy-3-methyl-1-propyl-imidazo[1,5-a]quinoxalin-4-yl)-methanesulfonamide;
N-(1-Cyclohexyl-8-cyclopropylmethoxy-3-methyl-imidazo[1,5-a]quinoxalin-4-yl)-methanesulfonamide;
N-[1-Cyclohexyl-3-methyl-8-(quinolin-2-ylmethoxy)-imidazo[1,5-a]quinoxalin-4-yl]-methanesulfonamide;
N-[1-(2-Chloro-phenyl)-7-methoxy-3-methyl-imidazo[1,5-a]quinoxalin-4-yl]-methanesulfonamide; and
N-(7-Methoxy-3-methyl-1-propyl-imidazo[1,5-a]quinoxalin-4-yl)-methanesulfonamide;
or a pharmaceutically acceptable salt thereof.

132. A method of preparing a compound of claim 82 wherein m and n are 0;
the bond between A and N is a double bond; and
R³ is CN,
comprising reacting a compound of formula (IV) 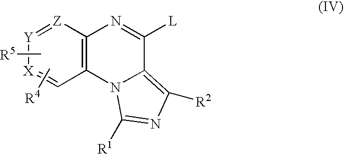
wherein L is Cl or Br; and R¹, R², R⁴, and R⁵ are as defined above with a cyanide salt.

133. The method of claim 132 wherein said cyanide salt is KCN.

134. A method of preparing a compound of claim 82,
wherein
m and n are 0;
the bond between A and N is a double bond;
R³ is selected from NHSO₂R⁸, N(SO₂R⁸)₂, N(R⁸)SO₂R⁸, NHSO₂R⁹, and N(R¹⁰)SO₂R⁹; and
R⁸, R⁹ and R¹⁰ are as defined in any one of claims 1-50,
comprising
(a) reacting a compound of formula (IV) 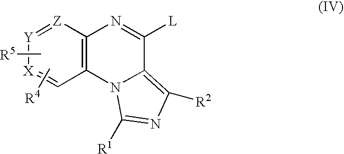
wherein L is Cl or Br; and R¹, R², R⁴, and R⁵ are as defined above;
with NH₃ or an alkyl amine to form a 4-amino derivative; and then
(b) reacting the 4-amino derivative with a sulfonic acid chloride or an anhydride to provide a final sulfonamide.

135. A pharmaceutical composition comprising as an active agent a compound of claim 82, or a pharmaceutically acceptable salt thereof, optionally together with a pharmaceutically acceptable carrier.

136. A method comprising administering to a subject in need thereof a therapeutically effective amount of the compound of claim 82, or a pharmaceutically acceptable salt thereof to treat or prevent a disorder associated with, accompanied by or caused by phosphodiesterase 10 hyperactivity or a disorder of phosphodiesterase 10.

137. A method comprising administering to a subject in need thereof a therapeutically effective amount of a compound of claim 82, or a pharmaceutically acceptable salt thereof, to treat or prevent a central nervous system disorder.

138. A method according to claim 137, wherein the disorder is schizophrenia, a psychotic disorder; a mood disorder; a neurotic disorder, a stress-related disorder, a somatoform disorder; an eating disorder; a sexual dysfunction comprising excessive sexual drive; a disorder of adult personality and behavior; a disorder usually first diagnosed in infancy, childhood and adolescence, mental retardation; a disorder of psychological development; a disorder having the symptom of a cognitive deficit; and a factitious disorder.

139. A method according to claim 137, wherein the disorder is episodic schizophrenia; a schizotypal disorder; a persistent delusional disorder; an acute psychotic disorder, a transient psychotic disorder, a persistent psychotic disorder; an induced delusional disorder; a schizoaffective disorder; a puerperal psychosis; or an unspecified nonorganic psychosis.

140. A method according to claim 137, wherein the disorder is a manic episode associated with bipolar disorder and single manic episode, hypomania, mania with psychotic symptoms; a bipolar affective disorder; a depressive disorder; a persistent mood disorder; or premenstrual dysphoric disorder.

141. A method according to claim 137, wherein the disorder is phobic anxiety, panic, or general anxiety disorder; obsessive compulsive disorder; a reaction to severe stress and adjustment disorder or a dissociative disorder.

142. A method according to claim 137, wherein disorder is a specific personality disorder of the paranoid, schizoid, schizotypal, antisocial, borderline, histrionic, narcissistic, avoidant, dissocial, emotionally unstable, anankastic, anxious and dependent type; a mixed personality disorder; a habit and impulse disorder; or a disorder of sexual preference.

143. A method according to claim 137, wherein the disorder is a hyperkinetic disorder, an attentional deficit/hyperactivity disorder (AD/HD), a conduct disorder; a mixed disorder of conduct and emotional disorder; a nonorganic enuresis, a nonorganic encopresis; a stereotyped movement disorder; an attention deficit disorder without hyperactivity, excessive masturbation, nail-biting, nose-picking, thumb-sucking; or a disorder of psychological development.

144. A method according to claim 137, wherein the disorder is a developmental disorder of speech and language, a developmental disorder of a scholastic skill which is predominantly diagnosed in infancy, childhood and adolescence.

145. A method according to claim 137, wherein the disorder is a cognitive deficit primarily but not exclusively related to psychosis; age-associated memory impairment, Parkinson's disease, Alzheimer's disease, multi infarct dementia, Lewis body dementia, stroke, frontotemporal dementia, progressive supranuclear palsy Huntington's disease and in HIV disease, cerebral trauma, drug abuse or mild cognitive disorder.

146. A method according to claim 137, wherein the disorder is a movement disorder with malfunction of basal ganglia selected from the group consisting of a focal dystonia, a multiple-focal or segmental dystonia, a torsion dystonia, a hemispheric, generalized and tardive dyskinesia, an akathisia, a dyskinesia selected from Huntington's disease, Parkinson's disease, Lewis body disease, restless leg syndrome, and PLMS.

147. A method according to claim 137, wherein the disorder is a symptomatic mental disorder; an organic delusional (schizophrenia-like) disorder; a presenil or senile psychosis associated to dementia, to psychosis in epilepsy and Parkinson's disease and other organic and symptomatic psychosis; delirium; infective psychosis; or a personality and behavioral disorder due to brain disease, damage and dysfunction.

148. A method according to claim 137, wherein the disorder is a mental and behavioral disorder due to psychoactive compounds, psychotic disorders, and residual and late-onset psychotic disorders induced by alcohol, an opioid, a cannabinoid, cocaine, hallucinogens, caffeine, volatile solvent or a psychoactive compound.

149. A method comprising administering to a mammal an effective amount of a compound of claim 82, or a pharmaceutically acceptable salt thereof a medicament for improving learning or memory capacity in the mammal.

150. A method comprising administering to a patient in need thereof a therapeutically effective amount of a compound of claim 82, or a pharmaceutically acceptable salt thereof to treat or prevent obesity, type 2 diabetes, metabolic syndrome, or glucose intolerance.

151. The method of claim 150, wherein said patient is overweight or obese.

152. The method of claim 150, wherein the compound is a selective PDE10 inhibitor.

153. The method of claim 150, wherein comprising administering a further therapeutic agent.

154. The method of claim 153, wherein said further therapeutic agent is an anti-obesity agent.

155. A method comprising administering to a patient a compound of claim 82, or a pharmaceutically acceptable salt thereof, to reduce body fat or body weight in the patient.

156. The method of claim 155, wherein said patient is overweight or obese.

157. The method of claim 155, wherein the compound is a selective PDE10 inhibitor.

158. The method of claim 155, further comprising administering to the patient a further therapeutic agent.

159. The method of claim 158, wherein said further therapeutic agent is an anti-obesity agent.

160. A pharmaceutical composition or kit which comprises at least one compound of claim 82, or a pharmaceutically acceptable salt thereof, in combination with at least one further pharmaceutically active compound.

161. The composition or kit of claim 160, wherein the further active compound is a therapeutically active compound useful in the treatment of central nervous system disorders which is not based on PDE 10 inhibition.
